(19) United States
(12) Patent Application Publication
(10) Pub. No.: US 2007/0265643 A1
(43) Pub. Date: Nov. 15, 2007

(54) Apparatus and method for suturelessly connecting a conduit to a hollow organ

(76) Inventors: Richard M. Beane, Hingham, MA (US); John W. Brown, Indianapolis, IN (US); James Alan Crunkleton, Weston, MA (US); James S. Gammie, Stevenson, MD (US); Joseph L. Smith JR., Concord, MA (US)

Correspondence Address: NIXON PEABODY, LLP, 401 9TH STREET, NW, SUITE 900, WASHINGTON, DC, 20004-2128, US

(21) Appl. No.: 11/783,287

(22) Filed: Apr. 6, 2007

Related U.S. Application Data
(63) Continuation-in-part of application No. 11/086,577, filed on Mar. 23, 2005.
(60) Provisional application No. 60/789,563, filed on Apr. 6, 2006.
(60) Provisional application No. 60/821,019, filed on Aug. 1, 2006.
(60) Provisional application No. 60/555,308, filed on Mar. 23, 2004.
(60) Provisional application No. 60/635,652, filed on Dec. 14, 2004.
(60) Provisional application No. 60/636,449, filed on Dec. 15, 2004.

(51) Int. Cl. A61B 17/08

(52) U.S. Cl. 606/153

(57) ABSTRACT

The present invention relates to an apparatus and method for securing a connector conduit to a hollow organ. The method comprises forming a hole in a wall of the organ; inserting a connector conduit through the hole in the wall of the organ until a flange element comes into contact with the wall of the organ, the flange element being positioned on the connector conduit; and engaging a retention means with the wall of the organ to prevent movement of the connector conduit relative to the wall of the organ, the retention means being positioned on the connector conduit. Exemplary retaining means include a plurality of retaining pins positioned circumferentially around the connector conduit, a plurality of prongs positioned circumferentially around the connector conduit, a balloon positioned on the connector conduit, a torsion spring positioned on the connector conduit, a spiral spring positioned on the connector conduit, or combinations thereof.

### RELATED APPLICATION DATA

This application is a continuation-in-part of U.S. patent application Ser. No. 11/086,577, filed Mar. 23, 2005, which claimed priority to U.S. Provisional Application Ser. Nos. 60/555,308, filed Mar. 23, 2004, 60/635,652 filed on Dec. 14, 2004, and 60/636,449 filed Dec. 15, 2004, and also claims priority to U.S. Provisional Application Ser. Nos. 60/789,563, filed Apr. 6, 2006, and 60/821,019, filed Aug. 1, 2006. The disclosures of each of the above applications are hereby incorporated by reference in their entirety.

### FIELD OF THE INVENTION

The present invention relates to an apparatus and method for securing a connector conduit to a hollow organ and preventing blood loss from the hollow organ, and more particularly, to a surgical device connectable to the apex of a heart.

### BACKGROUND

As the average age of the United States population increases, so do the instances of aortic stenosis. An alternative approach to the conventional surgical replacement of the stenotic aortic valve involves the use of an apicoaortic conduit. In this approach, the native aortic valve is not removed, and a prosthetic valve is implanted in a parallel flow arrangement. A connection conduit (or tube) connects the apex of the heart to the descending aorta. Somewhere along this conduit, the prosthetic valve is interposed. Thus, blood leaves the heart through the apex and travels through the conduit (with valve) to the descending aorta.

Until recently, surgical procedures to implant an apicoaortic conduit have included a single, long incision, such as in the 6^{th} intercostal space, to expose the heart and allow retraction of the lungs to expose the descending aorta. Recognizing the potential for broader scale use of the apicoaortic conduit for aortic valve replacement, some surgeons are now attempting to use smaller incisions and are requesting development of surgical tools for a minimally invasive procedure. As an initial attempt to make the procedure less invasive, some surgeons have recently performed the following procedure.

The patient is placed on the table in the supine position. Anesthesia is induced, and the patient is intubated with a double-lumen endotracheal tube, this facilitates one-lung ventilation and allows the surgeon to work within the left chest. The patient is positioned with the left side up (90 degrees). The pelvis is rotated about 45 degrees, such that the femoral vessels are accessible. An incision is made over the femoral vessels, and the common femoral artery and vein are dissected out. Heparin is administered. Pursestring sutures are placed in the femoral artery and vein. The artery is cannulated first, needle is inserted into the artery, and a guidewire is then inserted. Transesophageal echo is used to ascertain that the wire is in the descending aorta. Once this is confirmed, a Biomedicus arterial cannula is inserted over the wire, into the artery (Seldinger technique). The arterial cannula is typically 19 or 21 French. Once inserted, the pursestring sutures are snugged down over tourniquets. A similar procedure is followed for the femoral vein. The venous cannula is usually a few French larger than the arterial cannula. Once both vein and artery are cannulated, the cannulae are connected to the cardiopulmonary bypass, and the capability to initiate cardiopulmonary bypass at any time is present.

A 1 cm incision is made in approximately the 7^{th} interspace in the posterior auxiliary line; the videoscope (10 mm diameter) is inserted, and the left chest contents viewed. The location of the apex of the heart is determined, and the light from the scope used to transilluminate the chest wall; this allows precise localization of the incision. The incision is then performed; it is essentially an anterior thoracotomy, typically in the 6^{th} interspace. Recent incisions have been about 10 cm long, but are expected to become smaller and smaller with time. A retractor is inserted and the wound opened gently. A lung retractor is used to move the (deflated) left lung cephalad. The descending aorta is dissected free from surrounding soft tissue to prepare for the distal anastomosis. This dissection includes division of the inferior pulmonary ligament. A pledgeted suture is placed on the dome of the diaphragm and positioned to pull the diaphragm toward the feet (out of the way). The pericardium is incised about the apex of the heart, and the apex is freed up and clearly identified.

On the back table, the apicoaortic conduit is prepared: a Medtronic 21 Freestyle valve is sutured to an 18 mm Medtronic apical connector. The valve is also sutured to a 20 mm Hemashield graft. The Dacron associated with the apical connector is pre-clotted with thrombin and cryoprecipitate. The assembly is brought to the field, and a measurement made from the apex of the heart to the descending aorta. The assembly is trimmed appropriately. A partial-occluding clamp is then placed on the descending aorta, and the aorta opened with a knife and scissors. The conduit (the end with the 20 mm Hemashield graft) is then sutured to the descending aorta using 4-0 prolene suture, in a running fashion. Once this is complete, the clamp is removed and the anastomosis checked for hemostasis. Blood is contained by the presence of the freestyle aortic valve. The apical connector is placed on the apex, and a marker is used to trace the circular outline of the connector on the apex, in the planned location of insertion. Four large pledgeted sutures (mattress sutures) of 2-0 prolene are placed; one in each quadrant surrounding the marked circle. The sutures are then brought through the sewing ring of the apical connector. A stab wound is made in the apex in the center of the circle, and a tonsil clamp is used to poke a hole into the ventricle. To date, bypass has been initiated at this point, but doing so may not be necessary. A Foley catheter is inserted into the ventricle, and the balloon expanded. A cork borer is then used to cut out a plug from the apex. The connector is then parachuted down into position. A rotary motion is necessary to get the connector to seat in the hole. The four quadrant sutures are tied, and hemostasis is checked. If there is a concern regarding hemostasis, additional sutures are placed. The retractor is removed, chest tubes are placed, and the wound is closed.

Surgical tools developed specifically to implant the apicoaortic conduit are expected to provide the means for a much less invasive procedure. The procedure is expected to be performed with a series of smaller thoracotomy incisions between the ribs, such as immediately over the apex of the heart. In addition to avoiding the median sternotomy, development of appropriate surgical tools is expected to avoid the need for cardiopulmonary bypass, so that the procedure can be performed on a beating heart. The diseased aortic valve does not need to be exposed or excised. The stenotic aortic valve is left in place and continues to function at whatever level it remains capable of, and the apicoaortic conduit accommodates the balance of aortic output.

The major obstacle to widespread adoption of this superior technique is the nearly complete lack of efficient devices to perform the procedure. Surgeons wishing to adopt the procedure must gather a collection of instruments from a variety of manufacturers. Often these instruments were created for quite different purposes, and the surgeon is forced to adopt them as required and manually manipulate them during a procedure.

### SUMMARY OF THE INVENTION

The present invention relates to an apparatus and method for securing a connector conduit to a hollow organ and preventing blood loss from the hollow organ.

A preferred apparatus of the invention comprises a connector conduit operable to be inserted through a hole in a wall of the organ, a flange element positioned on the connector conduit adapted to prevent over-insertion of the connector conduit, and a retention means positioned on the connector conduit, the retention means being adapted to be engaged with the wall of the organ to prevent movement of the connector conduit relative to the wall of the organ after the connector conduit is inserted through the hole in the wall of the organ. The connector conduit is inserted through the hole in the wall of the organ until the flange element comes into contact with the wall of the organ, and the retention means is engaged with the wall of the organ after the connector conduit is inserted through the hole in the wall of the organ. The hole in the wall of the organ (i.e. a heart) may be formed by a hole forming element having a cutting element on a distal end thereof and being adapted for coupling with the connector conduit, and the flange element may be integrally formed on the connector conduit.

Similarly, a preferred method of the invention relates to a method for securing a connector conduit to a hollow organ, the method comprising forming a hole in a wall of the organ, inserting a connector conduit through the hole in the wall of the organ until a flange element comes into contact with the wall of the organ, the flange element being positioned on the connector conduit, and engaging a retention means with the wall of the organ to prevent movement of the connector conduit relative to the wall of the organ, the retention means being positioned on the connector conduit.

According to one embodiment of the invention, the retention means may comprise a plurality of retaining pins positioned circumferentially around the connector conduit, such that the retaining pins are inserted into the hole in the wall of the organ when the connector is inserted through the hole in the wall of the organ. In this configuration, the apparatus may include a means for causing the retaining pins to engage the wall of the organ to prevent movement of the connector conduit relative to the wall of the organ. The means for causing the retaining pins to engage the wall of the organ to prevent movement of the connector conduit relative to the wall of the organ may comprise a plurality of skid elements and pull wires, for example. In addition, the retaining pins are preferably maintained in a passive state adjacent to an outer surface of the connector conduit until entering into engagement with the wall of the organ.

According to another embodiment of the invention, the retention means may comprise a plurality of prongs positioned circumferentially around the connector conduit such that the prongs, when in an initial passive state, are positioned outside of the organ after the connector conduit has been inserted through the hole in the wall of the organ. In this configuration, after the connector conduit has been inserted through the hole in the wall of the organ, the prongs are adapted to be inserted through a plurality of holes in the flange element into the wall of the organ, thereby entering into engagement with the wall of the organ. A prong installation element may be used which is adapted to insert the prongs through the holes in the flange element into the wall of the organ, thereby causing the prongs to enter into engagement with the wall of the organ. The prongs may have a curved shape that causes engagement of the prongs with the wall of the organ by the insertion of the prongs into the wall of the organ.

According to a further embodiment of the invention, the retention means may comprise a balloon positioned on the connector conduit, such that the balloon is inserted through the hole in the wall of the organ as the connector conduit is inserted through the hole in the wall of the organ. The balloon is preferably maintained in an initial deflated state until after the balloon and the connector conduit are inserted through the hole in the wall of the organ. After the connector conduit has been inserted through the hole in the wall of the organ, the balloon may be inflated from the initial deflated state to an expanded state, thereby entering into engagement with the wall of the organ and preventing movement of the connector conduit relative to the hole in the wall of the organ. In addition, the flange element may be replaced with a second balloon positioned on the connector conduit such that, after insertion of the connector conduit through the hole in the wall of the organ, the two balloons are inflated, and the wall of the organ is compressed between the two balloons, thereby preventing movement of the connector conduit relative to the wall of the organ. Similarly, the flange element may be replaced with a torsion spring positioned on the connector conduit, such that, after insertion of the connector conduit through the hole in the wall of the organ, the balloon is inflated, and the wall of the organ is compressed between the torsion spring and the balloon, thereby preventing movement of the connector conduit relative to the wall of the organ.

According to a further embodiment of the invention, the retention means may comprise a torsion spring positioned on the connector conduit, such that the torsion spring, when in an initial compressed state, is inserted through the hole in the wall of the organ as the connector conduit is inserted through the hole in the wall of the organ. In this configuration, a sheath may be used to retain the torsion spring in a compressed state. After the connector conduit has been inserted through the hole in the wall of the organ, the sheath may be withdrawn from the hole in the wall of the organ, thereby allowing the torsion spring to expand from the initial compressed state to an expanded state, thereby entering into engagement with the wall of the organ and preventing movement of the connector conduit relative to the wall of the organ. The flange element may be replaced with a second torsion spring positioned on the connector conduit such that, after insertion of the connector conduit through the hole in the wall of the organ, and withdrawal of the sheath from the wall of the organ, the two torsion springs are in their respective expanded states, and the wall of the organ is compressed between the two torsion springs, thereby preventing movement of the connector conduit relative to the wall of the organ. Furthermore, the flange element may be replaced by a plurality of torsion springs positioned on the connector conduit such that, after insertion of the connector conduit through the hole in the wall of the organ, and withdrawal of the sheath from the wall of the organ, at least one torsion spring resides inside the organ, at least one torsion spring resides within the wall of the organ, and at least one torsion spring resides outside of the organ, thereby compressing the wall of the organ between the two torsion springs and preventing movement of the connector conduit relative to the wall of the organ. Also, the flange element may be replaced with a balloon positioned on the connector conduit such that, after insertion of the connector conduit through the hole in the wall of the organ, withdrawal of the sheath from the wall of the organ, and inflation of the balloon, the torsion spring is in its expanded state, the balloon is in its inflated state, and the wall of the organ is compressed between the torsion spring and the balloon, thereby preventing movement of the connector conduit relative to the wall of the organ.

According to a further embodiment of the invention, a spiral spring may be positioned on the connector conduit, such that the spiral spring, when in an initial compressed state, is inserted through the hole in the wall of the organ as the connector conduit is inserted through the hole in the wall of the organ. In this configuration, a smooth frame cover may be used to retain the spiral spring in a compressed state. After the connector conduit has been inserted through the hole in the wall of the organ, the smooth frame cover can be withdrawn from the hole in the wall of the organ, thereby allowing the spiral spring to expand from the compressed state to an expanded state, thereby entering into engagement with the wall of the organ and preventing movement of the connector conduit relative to the wall of the organ. The flange element may be replaced by a compression ring, which is positioned circumferentially around the connector conduit on the outside of the organ, such that, after the connector conduit is inserted through the hole in the wall of the organ, the spiral spring expands from the compressed state to an expanded state, and the compression ring is moved longitudinally along the surface of the connector conduit along one or more ratchet steps formed on the surface of the connector conduit towards the wall of the organ, thereby compressing the wall of the organ between the spiral spring and the compression ring, and preventing movement of the connector conduit relative to the wall of the organ.

Thus, the present invention provides an apparatus and method that may be used by a surgeon in accordance with connector conduit and applicator systems, such as those disclosed in U.S. patent application Ser. No. 11/086,577 filed Mar. 23, 2005 and Ser. No. 11/300,589 filed Dec. 15, 2005, and U.S. Provisional Patent Application Nos. 60/726,222 and 60/726,223, both filed Oct. 14, 2005, the disclosures of which are hereby incorporated by reference in their entirety. The securing means of the present application may be used, for example, with any type of suitable system, such as the system of the '577 application.

### BRIEF DESCRIPTION OF THE DRAWINGS

FIG. 1 illustrates an apicoaortic conduit.

FIG. 2A is a cross-sectional view another embodiment of the structural frame of the connector, covered in fabric, with an incorporated sewing flange and shown in the bent configuration.

FIG. 2B is a cross-sectional view of the structural frame of the connector of FIG. 3A shown in a straight configuration.

FIG. 2C is a cross-sectional view of the connector of FIG. 2A shown in the straight configuration, and with a fabric conduit in place.

FIG. 3 is a cross-sectional view of an embodiment of the device showing the coring element and the retractor element in place within the straightened connector.

FIG. 4 is a cross-sectional view of a cylinder plug tool that slides over the retractor element and into the coring element, which is used to load the connector-conduit onto the coring element.

FIG. 5 is a cross-sectional view of an embodiment of the device showing the placement of a compression spring between the retractor element and the coring element.

FIG. 6 is a cross-sectional view of another embodiment of the device showing the placement of a pushing element.

FIG. 7A is a cross-sectional view of yet another embodiment of the device showing the attachment of a handle to the pushing element with an access means for the expandable element integrated into the pushing element, wherein the expandable element is shown contracted.

FIG. 7B shows the embodiment of FIG. 7A with the expandable element expanded.

FIG. 8 is a cross-sectional view of an embodiment of the device showing the inclusion of a sliding bolt on the retractor element and related indexed slots on the pushing device.

FIG. 9 is a partial view the pushing element of FIG. 8 showing the indexed slots on the pushing device.

FIG. 10A is a perspective view of a flexible structural frame of another embodiment of the connector conduit shown in a straight configuration.

FIG. 10B is a perspective view of the structural frame of FIG. 10A shown in a bent configuration.

FIG. 10C is a perspective view of the structural frame of FIG. 10B shown with a beveled and tapered leading edge.

FIG. 11 is a perspective view of an alternative embodiment of FIG. 9B.

FIG. 12A is a perspective view of the flexible structural frame of FIG. 10B shown in the straightened configuration and incorporating a bending means.

FIG. 12B is a perspective view of the structural frame of FIG. 12A after activating the bending means.

FIG. 13 is a perspective view of a non-bendable structural frame of a connector conduit.

FIG. 14 is a cross-sectional view of a connector conduit shown in a bent configuration.

FIG. 15 is a cross-sectional view of a non-bendable connector conduit.

FIG. 16A is a cross-sectional view of a mounting element (including a coring element) and a pushing element of the applicator with a loaded connector conduit.

FIG. 16B is a cross-sectional view FIG. 16A without the connector conduit.

FIG. 17A is a perspective view of a squeeze ring for a locking means to secure the connector conduit within the applicator.

FIG. 17B is a perspective view of a locking means shown in the locked position.

FIG. 17C is a perspective view of a locking means shown in the unlocked position.

FIG. 18 is a cross-sectional view of the device of FIG. 16B including a retractor element.

FIG. 19 is a cross-sectional view of a folding and mounting tool.

FIG. 20 is a cross-sectional view of an assembly including an applicator having a syringe.

FIG. 21A is a cross-sectional view of a sequencing bolt.

FIG. 21B is a cross-sectional view of the retractor body and expanding element.

FIG. 21C is a cross-sectional view of the positioning mans and coring element.

FIGS. 22A-22C the sequencing can mechanism in various states.

FIGS. 23A-23E illustrate the applicator in various states.

FIG. 24 is a perspective view of an integrated connector conduit and cutting elements.

FIG. 25 is the device of FIG. 24 with the cutting element withdrawn.

FIGS. 26A-26D illustrate components of a retractor having an expandable umbrella element.

FIGS. 27A-27E illustrate an embodiment of the invention wherein the connector conduit is attached to the organ using one or more retaining pins.

FIGS. 28A-28E illustrate an embodiment of the invention wherein the connector conduit is attached to the organ using one or more prongs.

FIGS. 29A-29D illustrate a prong deployment mechanism operable to install the prongs illustrated in FIGS. 28A-28E.

FIGS. 30A-30C illustrate an embodiment of the invention wherein a balloon is used to retain the connector conduit securely within the organ.

FIGS. 31A-31B illustrate an exemplary balloon that may be used in the system of FIGS. 30A-30B.

FIGS. 32A-32C illustrate an embodiment of the invention wherein a torsion spring is used to retain the connector conduit securely within the organ.

FIGS. 33A-33C illustrate an embodiment of the invention wherein a spiral spring is used to retain the connector conduit securely within the organ.

### DETAILED DESCRIPTION OF THE PREFERRED EMBODIMENTS

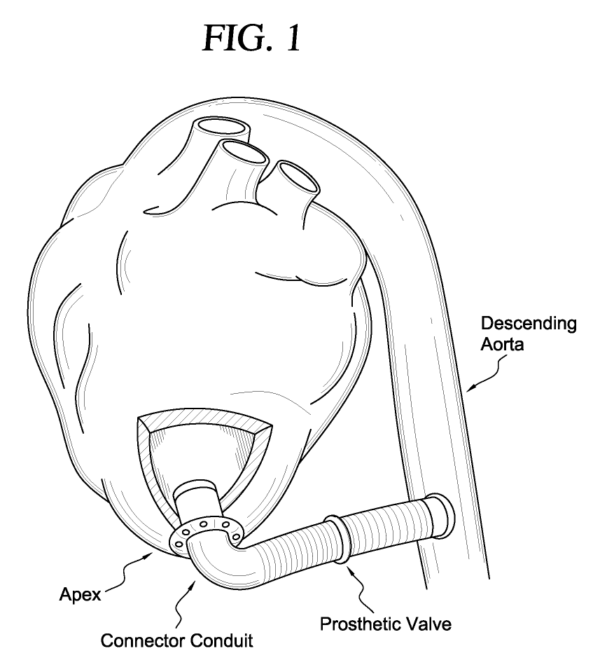

FIG. 1 is an illustration of an apicoaortic conduit, which extends from the apex of the left ventricle to the descending aorta with a prosthetic valve positioned within the conduit. The preferred embodiment of the present invention includes aspects of the connector conduit and an applicator used to implant the connector conduit.

The connector-conduit with applicator of the present invention is best described as consisting of five major parts: a connector-conduit, a retractor, hole forming device such as a coring element, a pushing component, and a handle. A fabric material pleated conduit of a type common and well known in the field is permanently fixed to the inner surface of a rigid connector to form the connector-conduit. The conduit extends from the forward edge of the connector and continues beyond the connector, as a flexible portion, for some distance.

The connector-conduit includes a rigid portion defined by an internal support structure made of a suitably flexible material that is preferentially biased to assume a bent configuration upon applying a bending force or that is preferentially biased to assume a bent configuration (such as a right angle) upon removal of restraining forces. In one embodiment, the connector internal support structure is covered with fabric, such as knitted or woven Dacron, for example. A suturing ring is integrated into the covering fabric and provides a suitable flange for suturing the connector to the surface of the heart. The leading edge of the connector is tapered to facilitate insertion of the connector-conduit component. The “rigid” portion is rigid enough to facilitate insertion as described below and to maintain the hole in an open position. However, the rigid portion can be flexible. Accordingly, the term “rigid” as defined herein means relatively rigid and can include flexibility.

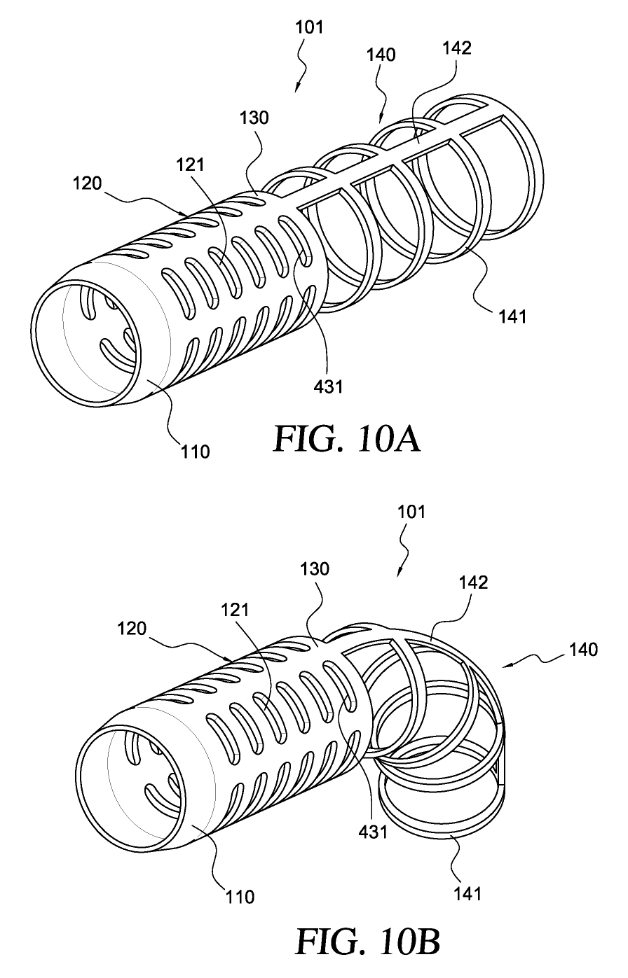

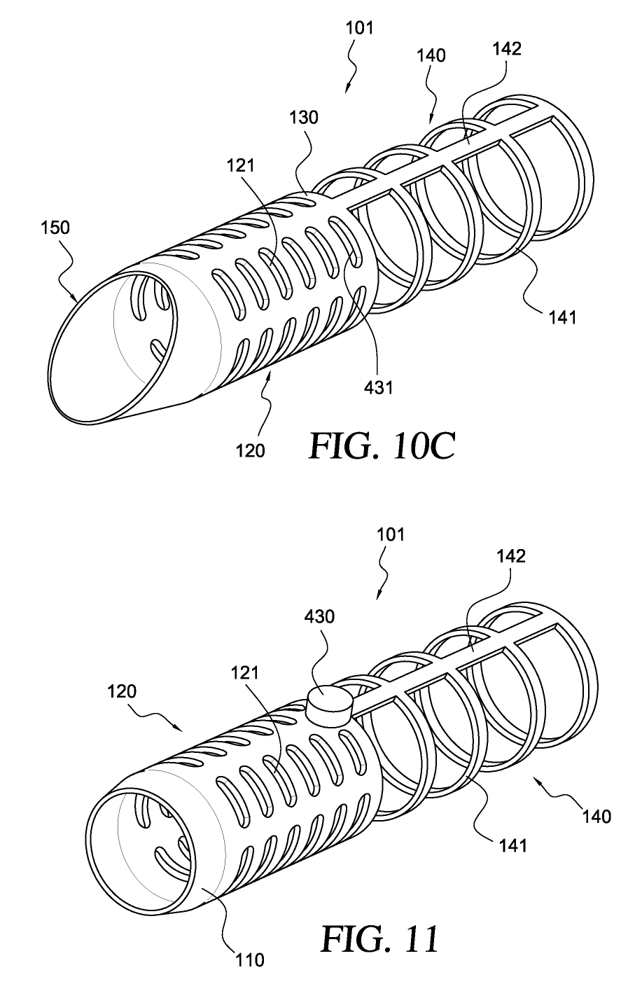

As shown in FIG. 10B, the structural frame 140 of the connector-conduit is a series of circular rings 141 joined to a curved spine 142. During implantation, the curved spine 142 is straightened, as shown in FIG. 10A, resulting in a straight pathway for the passage of instruments. As an alternative, the connector-conduit could include circular rings 141 without curved spine 142. As such, the circular rings would prevent collapse of the conduit, but the curved conduit would be formed manually after implantation, rather than by being formed by the curved spine 142. As another alternative, a modified coil spring in the shape of a curve could be used instead of circular rings 141 and curved spine 142. Properties of the coil spring would be chosen to prevent radial collapse and to provide appropriate stiffness of the curved position.

The leading edge of structural frame 101 is a tapered leading edge 110 which allows for easy insertion of the connector through the ventricle wall. The material of the structural frame 101 could be a shape memory alloy (e.g., Nitinol), plastic, or other similar biocompatible material.

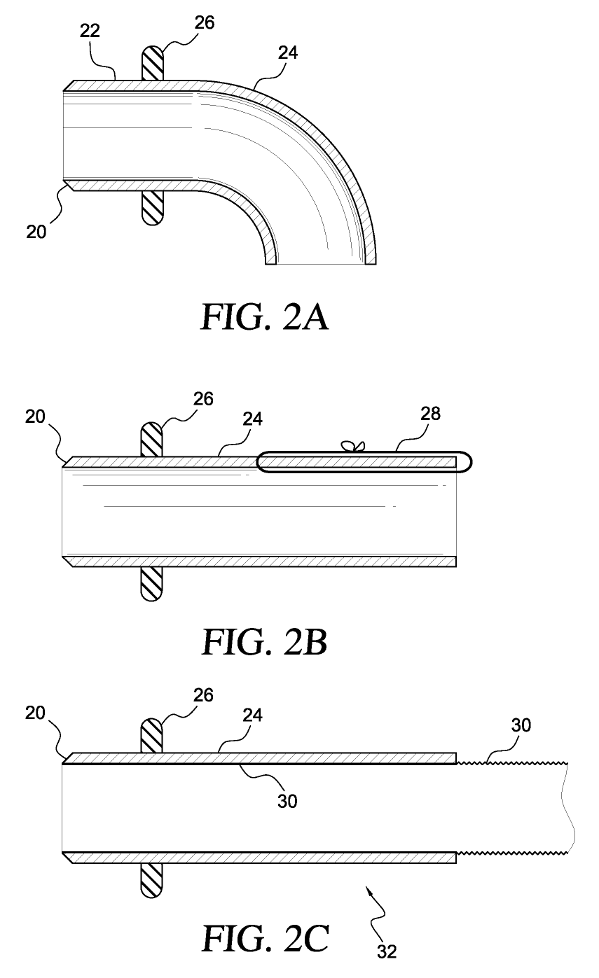

FIG. 2A illustrates a fabric covering 24 over the outside surface of structural frame 101 (not shown). Because connector surface 22 is in contact with the myocardial hole after implantation, a suturing ring or flange 26 is incorporated into the fabric covering 24 to provide an attachment site for sutures to anchor the connector to the heart. The fabric covered suture ring 26 could be made of a biocompatible foam or rubber.

FIG. 2B shows the fabric covered structural frame 101 (not shown) and suturing flange 26 in a straightened position. The straightened position can be achieved by, for example, inserting a straight instrument through the lumen of the frame. Alternately, the structure can be held in the open position through the use of stay stitches 28, or the like, placed such that the circular rings 141 (not shown) are held in close proximity.

FIG. 2C is a view similar to FIG. 2B, showing the structural frame in the straightened position with a pleated fabric conduit 30. Conduit 30 extends from tapered leading edge 110 of the structural frame 101 (not shown), through the length of the structural frame 101, and for some additional length beyond the structural frame 101 to define a flexible portion of the connector conduit. An orientation marker (not shown) on connector surface 22, for example, is used to identify the direction that conduit 30 will be oriented once implanted into the heart. The orientation marker is visible at all times to assist the surgeon while placing the connector-conduit 32 into the connector-conduit applicator and to facilitate implantation at an appropriate angle into the heart. Also, a radiopaque marker(s) (not shown) may be integrated into the entire length of fabric covering 24 and conduit 30 to facilitate identification and location of the structure by X-ray or other means.

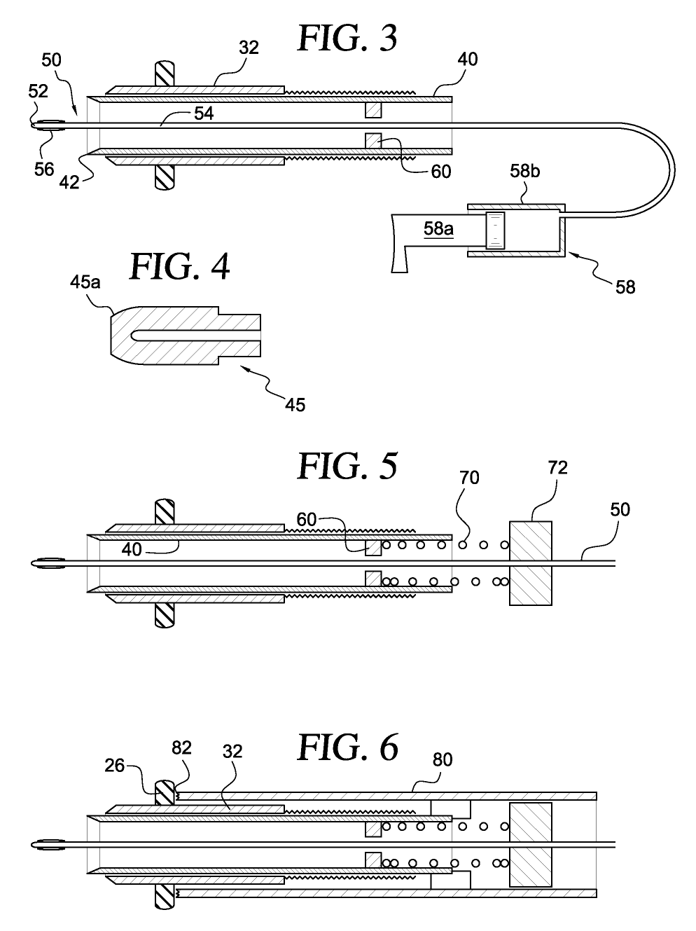

Referring to FIG. 3, in accordance with another embodiment of the present invention, a hole forming device such as coring element 40, is placed concentrically within the lumen of the connector-conduit 32. The coring element 40 preferably consists of a tubular structure, which could be made entirely of metal (such as stainless steel) or primarily of a plastic material with a metal insert for the leading edge 42. In a preferred configuration, the leading edge 42 of coring element 40 may be suitably sharpened such that it cuts a plug of tissue of approximately the same diameter as the outer diameter of the coring element 40. Note that the hole forming device can be any known mechanism for forming a hole, such as a laser cutter, a thermal ablation device, a chemical ablation device, or the like.

An interference fit between connector surface 22 and the hole created by the coring element 40 is necessary to reduce bleeding from the cut myocardial surface and to reduce blood leakage from the left ventricle. The amount of such interference fit is the difference between the diameters of the hole created by the coring element 40 and the outer surface of the connector 22.

In a preferred embodiment of the device, the coring element 40 has an outer diameter that closely matches the inner diameter of the connector-conduit 32. Such construction allows removal of the coring element 40 through the connector-conduit 32 while presenting only a small blood pathway between these two elements. Such construction is intended to minimize blood loss from the left ventricle when the coring element 40 has completed its cut.

FIG. 3 further illustrates the concentric placement of the retractor element 50 within the coring element 40. Retractor element 50 includes a blunt tip 52, a tubular body 54, an expanding element 56, such as a balloon, and an access means 58 for engageably expanding element 56. Access means 58 can be a plunger 58a in a cylinder 58b configuration, whereby displacement of the plunger expands or contracts expanding element 56. A centering plug 60 is shown concentrically positioned within and rigidly attached to coring element 40. The centering plug 60 concentrically positions retractor element 50, which slideably moves within the centering plug 60. The centering plug 60 also presents a barrier to the flow of blood through coring element 40, once the tissue plug is formed. Proper placement of centering plug 60 within coring element 40 should consider tradeoffs between two different parameters. First, centering plug 60 should be placed at a position within coring element 40, which allows ample space for the expanding element 56 and the tissue plug. Second, since radial force from the heart wall tends to deflect the expanding element 56, retractor element 50 must have a sufficient stiffness to substantially resist such deflection. Such deflection may also be reduced by limiting the axial distance between the expanding element 56 and centering plug 60.

FIG. 4 shows a cylinder plug tool 45 for insertion into coring element 40 prior to loading connector-conduit 32 onto coring element 40. Cylinder plug tool 45 facilitates loading connector-conduit 32 without damage from leading edge 42 of coring element 40. Once the connector-conduit 32 is loaded, cylinder plug tool 45 is removed and placed aside. As a safety measure, cylinder plug tool 45 has an extended length with a tapered blunted end 45a, which extends to cover retractor element 50, preventing insertion of the retractor element 50 into the left ventricle before cylinder plug 45 is removed.

Referring to FIG. 5, another embodiment of the present invention shows a compression spring 70 placed around the retractor element 50. One end of the compression spring 70 seats on the centering plug 60, and the other end seats on a sliding plug 72. Sliding plug 72 is rigidly connected to retractor element 50. Spring 70 ensures that expanding element 56 seats snugly against the inside wall of the ventricle to symmetrically displace the ventricle wall from the path of the coring element. Once the tissue plug is cut from the ventricle by coring element 40, spring 70 also pulls the tissue plug fully within the coring element 40.

FIG. 6 illustrates a further embodiment, wherein a cylinder-shaped pushing element 80 is positioned concentrically outside the connector-conduit element 32. Pushing element 80 is used to apply force to the coring element 40 and connector-conduit element 32. This force is required for the coring element 40 to cut the hole in the myocardium and for pushing the connector-conduit element 32 into the hole. The end of the pushing element 80 that is in contact with the suture ring 26 has a roughened surface 82 intended to prevent relative rotary motion between the suture ring 26 and pushing element 80. As such, the pushing element 80 allows both a force and a back-and-forth rotary motion to simultaneously be applied to the coring element 40 and connector-conduit element 32, as required to fully seat the suture ring 26 flush with the surface of the heart. Pushing element 80 could be made of metal, plastic or other suitable material.

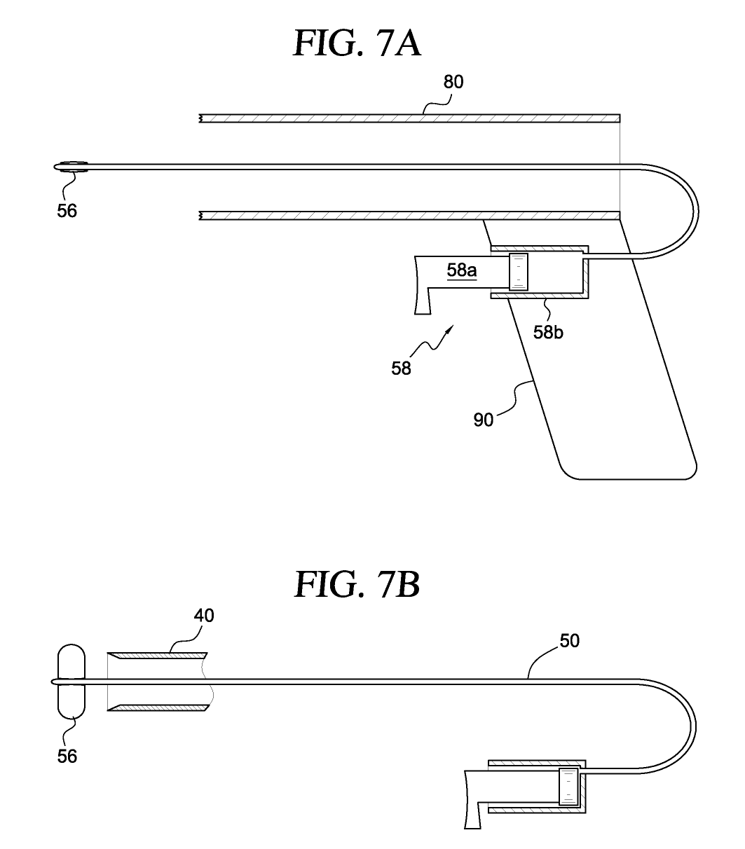

Referring to FIGS. 7A and 7B, a handle 90 is rigidly attached to pushing element 80. As shown, handle 90 is configured similar to a pistol grip, for example, handle 90 having an angle of about 70 degrees, with the pushing element 80. Handle 90 provides a user-friendly interface for the surgeon to hold with one hand, to position the coring element 40, to apply axial force to the connector-conduit element and to provide a back-and-forth rotational motion of around 90 degrees. Of course, many alternatives exist for the user interface. For example, the pushing element 80 itself could be used as the handle. As another example, a handle could form a “T” shape on the end of the pushing element 80.

Also shown in FIG. 7A, an access means 58 is used to expand or contract expanding element 56. Access means 58, for example, can be a trigger-type mechanism integrated into handle 90. As such, the user can use a finger to pull plunger 58a into the cylinder 58b, thereby displacing the fluid (such as saline) inside the cylinder 58b into the balloon 56. FIG. 7B shows the inflation of the balloon 56. As a safety feature, the plunger can have a latching device (not shown) that latches the plunger 58a with the balloon fully inflated, thereby preventing deflation of the balloon before intended.

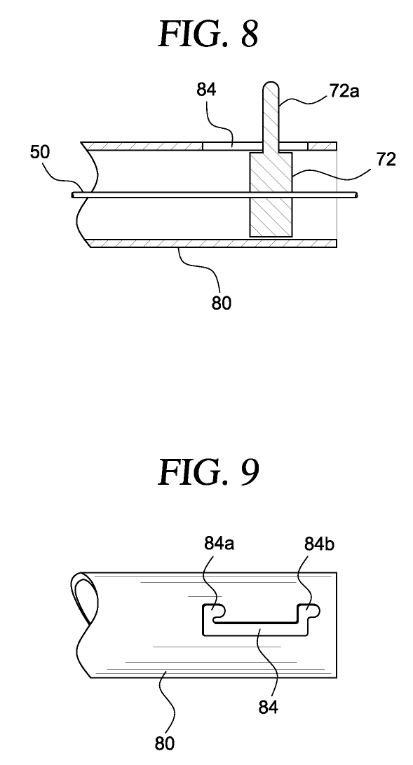

FIGS. 8 and 9 show a mechanism for controlling deployment of the retractor element 50. A slot 84 is cut into pushing element 80. Slot 84 has an index 84a to lock retractor element 50 at full extension and an index 84b to lock retractor element 50 at full retraction. Bolt 72a is rigidly attached to sliding plug 72. Bolt 72a can be manually displaced within slot 84 to position the retractor element 50. In operation, bolt 72a is positioned in index 84a until the retractor element 50 is fully inserted into the left ventricle and the expanding element 56 is at full expansion. At that time, bolt 72a is manually released from index 84a, which allows compression spring 70 to retract retractor element 50 until expanding element 56 contacts the inside wall of the left ventricle. A damping means (not shown) may be included to prevent sudden retraction of the retractor element upon release from index 84a. Also not shown is a safety latch or other means to prevent manual release of the bolt 72a until the expanding element 56 is fully expanded.

As the surgeon applies force and rotation using handle 90, compression spring 70 continues to displace retractor element 50. When retractor element 50 is fully retracted, the surgeon can rotate bolt 72a into index 84b to lock the retractor element 50 in place. Moreover, when retractor element 50 is fully retracted, the expanding element 56 is also fully retracted into coring element 40, indicating that the tissue plug has been successfully removed from the left ventricle and is within the coring element 40.

Referring to the embodiment of FIGS. 10A-10C, the connector conduit has a structural frame 101 defining a rigid portion, which may be constructed from a single material or a combination of materials. The structural frame 101 includes a tapered leading edge 110 designed to reduce the effort needed to push the connector through the heart wall located at one end of a cage section 120 and a bend portion 140 that is normally biased into a bent configuration. As shown in FIG. 10C, a tapered and beveled leading edge 150 may further reduce the required effort. During use, cage 120 resides primarily within the heart wall, so it must be constructed so as to be rigid enough to not collapse due to radial forces exerted by the heart wall. The cage 120 may include cage slots 121. The cage slots 121 allow the passage of thread to secure the conduit or the sewing flange.

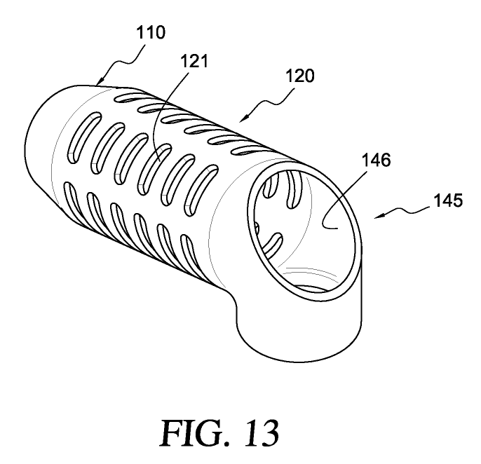

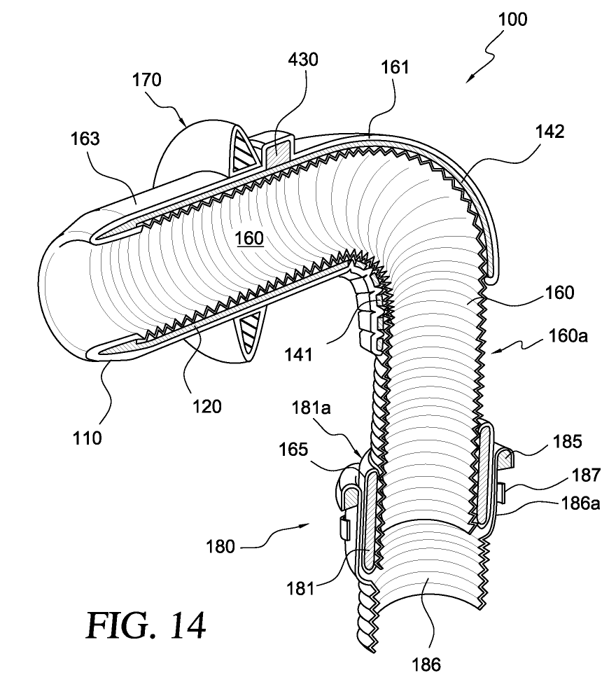

A holder 130 is formed at one end of cage 120 and may be used to grasp the connector during implantation. As will be described further herein, holder 130 can have a slot-and-key configuration with the applicator. As such, the holder 130 utilizes holder slots 431 or a holder button 430 (FIG. 11). Holder button 430 may be a separate part that is anchored (e.g., by thread or glue) to structural frame 101. If desired, the holder slots 431 or holder button 430 may be designed to place the flexible bend 140 or rigid bend 145 (FIG. 13) at a preferred angle relative to the applicator. Alternatively, the holder 130 may rely upon a tight friction fit with the applicator. In a preferred configuration, the holder 130 relies upon both a slot-and-key and a tight friction fit to lock the holder 130 relative to the applicator.

Referring again to FIGS. 10A and 10B, bend portion 140 includes circular rings 141 and a curved spine 142. The circular rings 141 prevent radial collapse of the conduit, and the curved spine 142 holds the conduit in a preferred shape to direct blood flow from the heart to the aorta. The curved spine 142 may be at the outer radius of bend portion 140 (as shown) or at the inner radius of the flexible bend. As an alternative, flexible bend 140 may include two curved spines at the mean radius. As another alternative, the structural frame 101 could include circular rings 141 without curved spine 142. As another alternative, a modified coil spring in the shape of a preferred bend could be used instead of circular rings 141 and curved spine 142. Properties of the coil spring would be chosen to prevent radial collapse and to provide appropriate stiffness of the curved position.

The structural frame of FIGS. 10A-11 is intended for mounting onto the outer diameter of a straight mounting element. As such, the bend portion 140 must be constructed to allow straightening of the curved spine 142. If curved spine 142 is made of a material or combination of materials with higher modulus of elasticity (e.g., PEEK, metal), the flexible bend 140 is stiffer. As such, the flexible bend 140 may be biased to resume a preferred shape (e.g., a 90° bend) when removed from the mounting element. If the curved spine 142 is made of a material with a lower modulus of elasticity (e.g., polypropylene, polyethylene), the bend portion 140 is less stiff. As such, the bend portion 140 may be biased relatively straight when removed from the straight mounting element. In such case, some bending means may be needed to position the bend portion 140 into the preferred shape.

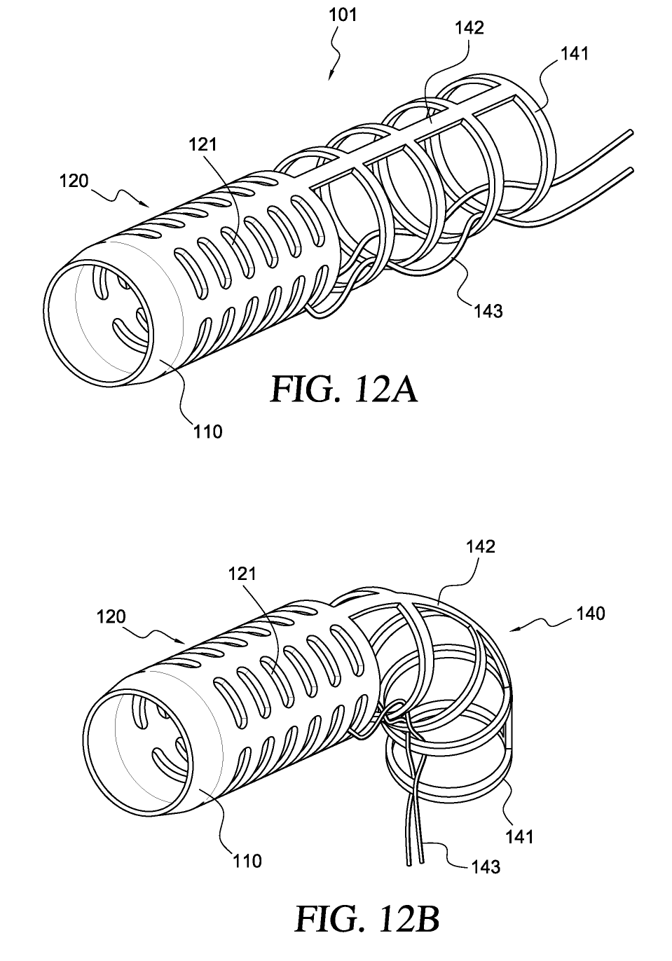

One embodiment of a bending means is shown in FIGS. 12A and 12B, which illustrate use of threads 143 that are secured to the holder 130 (for example) and weaved through circular rings 141. When threads 143 are pulled, the bend portion 140 changes from the normally biased, straight configuration of FIG. 12A to the bent configuration of FIG. 12B. When the flexible bend 140 reaches the preferred shape, the threads may be tied to form a knot or crimped. If desired, the bending means can be used with a curved spine 142 constructed of a high modulus of elasticity material to prevent straightening beyond the preferred angle.

As discussed previously, structural frame 101 may be constructed with a fixed bend 145, as shown in FIG. 13. A port 146 allows the mounting of structural frame 101 with a fixed bend 145 onto a straight mounting element.

FIG. 14 is a cross-section of a connector conduit 100 that includes a rigid portion defined by structural frame 101 with bend portion 140, and a flexible portion defined by conduit 160. The rigid portion also includes outer fabric 161, and sewing flange 170. Orientation marks (not shown) may be included on the conduit 160 or outer fabric 161. Conduit 160 may be a pleated vascular graft constructed of woven Dacron. Outer fabric 161 could be a knitted Dacron fabric material that stretches to accommodate contours of the structural frame 101. Sewing flange 170 could be constructed of a soft silicone rubber, for example, to allow easy passage of a needle when fastening sewing flange (or sewing ring) 170 to the outer surface of the heart. To allow visualization on x-ray, for example, the sewing flange could be made radiopaque, such as by mixing barium sulfate into the silicone rubber. The sewing flange may have a cloth covering such as that used for outer fabric 161. Alternatively, the sewing flange 170 may consist entirely of folded cloth. The components of the connector conduit 100 may be fastened together as needed, such as with thread.

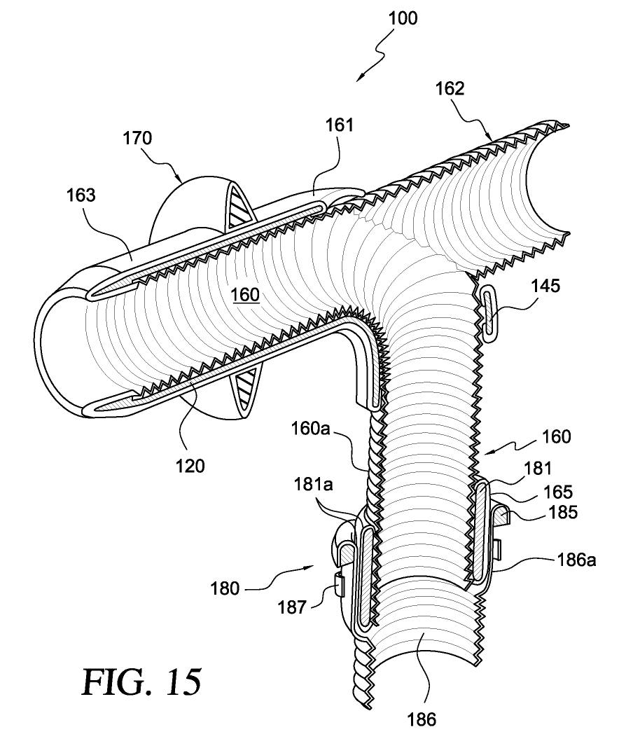

Referring to FIG. 15, a cross-section of a connector conduit 100 is similar to that shown in FIG. 14, except that the structural frame 101 is constructed with fixed bend 145. A conduit branch 162 intersects with conduit 160 through port 146 of rigid bend 145 to allow passage of a straight mounting element through the connector conduit 100. Once the connector conduit 100 is implanted into the ventricle, branch 162 may be occluded at the intersection with conduit 160. Branch 162 may then be cut off.

FIG. 14 and FIG. 15 further illustrate a quick connect coupler 180 for expediting attachment of the connector conduit 100 to the remainder of the prosthesis, which may include a prosthetic valve or ventricular assist device, as examples. As shown, the male end of quick connect coupler 180 is a continuation of or is attached to vascular graft 160. The male end of quick connect coupler 180 includes rigid connector frame 181, which may be constructed of a biocompatible plastic or metal. Vascular graft 160 covers the inner diameter of connector frame 181, and an outer fabric 165 covers the outer diameter of connector frame 181. Outer fabric 165 may be continuous with vascular graft 160. Outer fabric 165 is not of a pleated construction, such as is typical of vascular graft 160. The cloth-covered connector frame 181 provides a rigid surface onto which the female end of quick connect coupler 180 may be mounted. The female end of quick connect coupler 180 includes vascular graft 186 and pull ring 185. Vascular graft 186 attaches on its downstream end to the remainder of the prosthesis, which may include a prosthetic valve or ventricular assist device, as examples. Vascular graft 186 may be a pleated vascular graft constructed of woven Dacron, for example. Graft extension 186a is a continuation portion of or is attached to vascular graft 186. A rigid pull ring 185 (which may be constructed of a biocompatible plastic or metal) is attached to graft extension 186a. The male end of quick connect coupler 180 has a larger outer diameter than vascular graft 186. This construction provides a stop so that the male end of quick connect coupler 180 reaches an abrupt change to a smaller diameter provided by vascular graft 186. In this way, the surgeon knows when the male end is fully inserted into the female end of quick connect coupler 180. In use, the surgeon may grasp pull ring 185 with one hand and connector frame segment 181 a of connector frame 181 with the other hand. Pull ring 185 is pulled over outer fabric 165 until the male end of quick connect coupler 180 contacts the smaller diameter vascular graft 186. A large suture or umbilical tape 187 may then be tied around graft extension 186a to reduce blood loss by occluding the annular gap between the outer diameter of outer fabric 165 and the inner diameter of graft extension 186a. Stay sutures may also be used to connect outer fabric 165 to graft extension 186a, thereby preventing separation of the male and female ends of quick connect coupler 180.

FIG. 14 and FIG. 15 further illustrate a collapsible portion 160a between connector conduit 100 and quick connect coupler 180. Such collapsible portion 160a allows use of a cross clamp, for example, to fully collapse portion 160a to occlude flow after the applicator is removed beyond collapsible portion 160a. Collapsible portion 160a can be made of the same material as the rest of the flexible portion, or can be made of a different material.

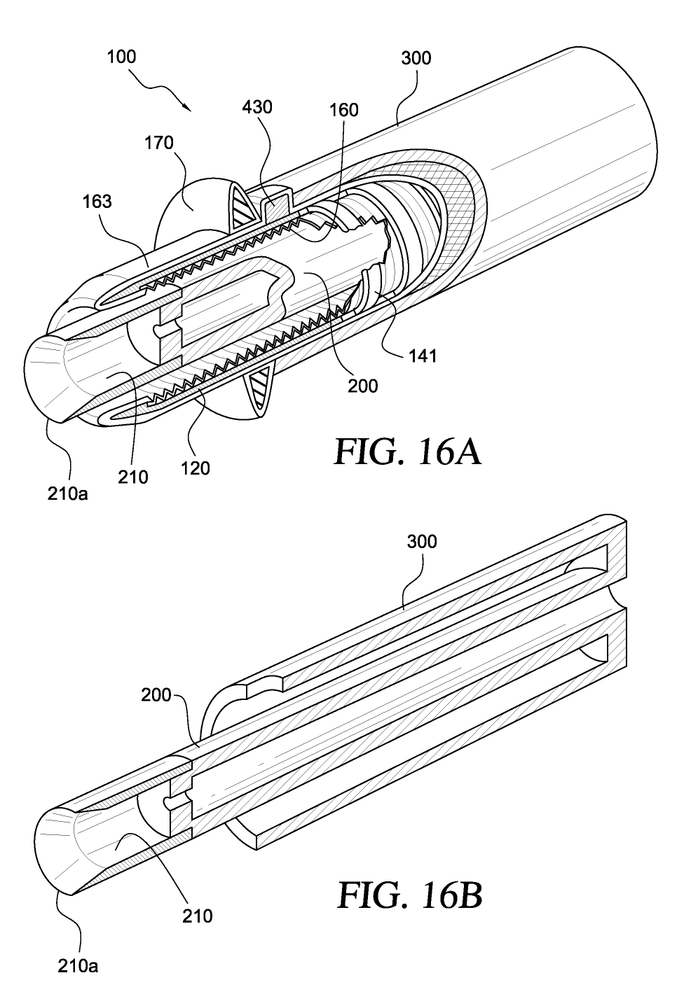

In use, the applicator of the present invention is used to implant the connector conduit 100 into the ventricle wall or other organ wall. FIG. 16A shows a cross-section of the connector conduit 100 (FIG. 14) loaded onto a mounting element 200. For clarity, the applicator is shown without the connector conduit 100 in FIG. 16B. Mounting element 200 includes a cylindrical coring element 210, serving as a hole forming element, that is concentric with and has the same diameter as the mounting element 200. The mounting element 200 and coring element 210 are placed concentrically within the lumen of the connector conduit 100. Coring element 210 includes a thin-walled tube and a sharpened cutting edge 210a, which may be tapered on the inner diameter, for example, to form the sharpened cutting edge 210a. The coring element 210 is used to cut a cylindrical-shaped core (or hole) in the heart wall, producing a plug from the heart wall that resides within the coring element 210. The mounting element 200 could be constructed of plastic (e.g., ABS), and the coring element 210 could be constructed of metal (e.g., stainless steel). In a preferred embodiment, the mounting element 200 and coring element 210 have an outer diameter that closely matches the inner diameter of the connector conduit 100. One purpose of such a construction is to minimize blood loss from the left ventricular chamber when the coring element 210 has completed its cut. Also in order to reduce blood loss from the left ventricular chamber and from the cut myocardial surface and to yield a snug fit of the connector conduit within the ventricular myocardium, the cutting diameter of the coring element 210 is chosen to produce a core that is smaller in diameter than the outer surface 163 of the of the connector conduit 100.

FIGS. 16A and FIG. 16B further illustrate a cylinder-shaped pushing element 300 positioned concentrically outside the connector conduit 100. In a preferred embodiment, the pushing element 300 transmits pushing force and rotation to the connector conduit 100. In further accordance with a preferred embodiment, the pushing element 300 is rigidly attached to mounting element 200, such that pushing element 300 transmits pushing force and rotation to the mounting element 200 and coring element 210. Pushing element 300 may be constructed of plastic (e.g., ABS) or metal (e.g., stainless steel). However, it should be appreciated that the present invention contemplates the use of other materials.

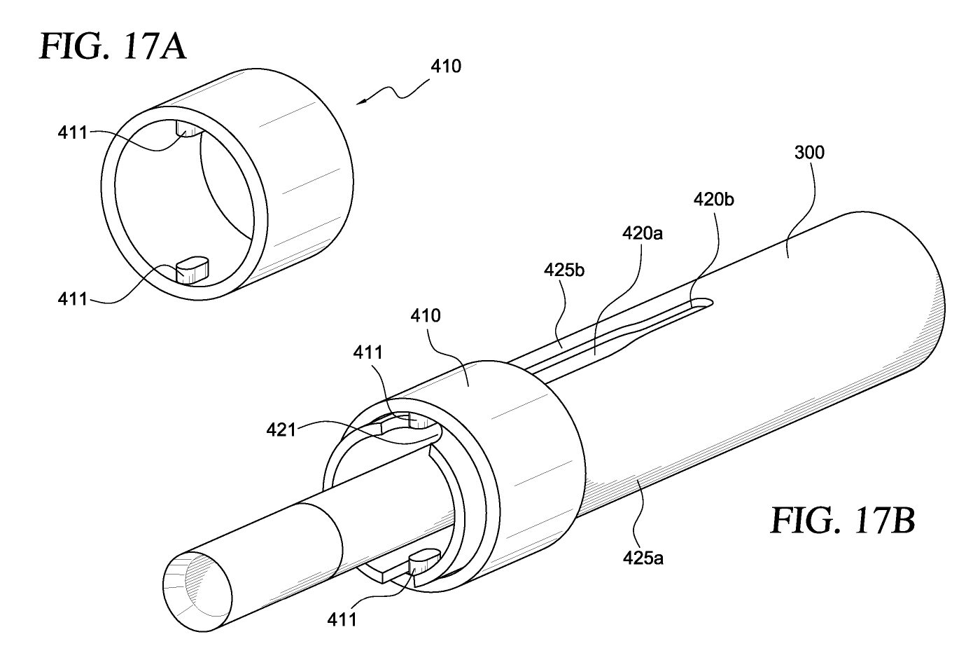

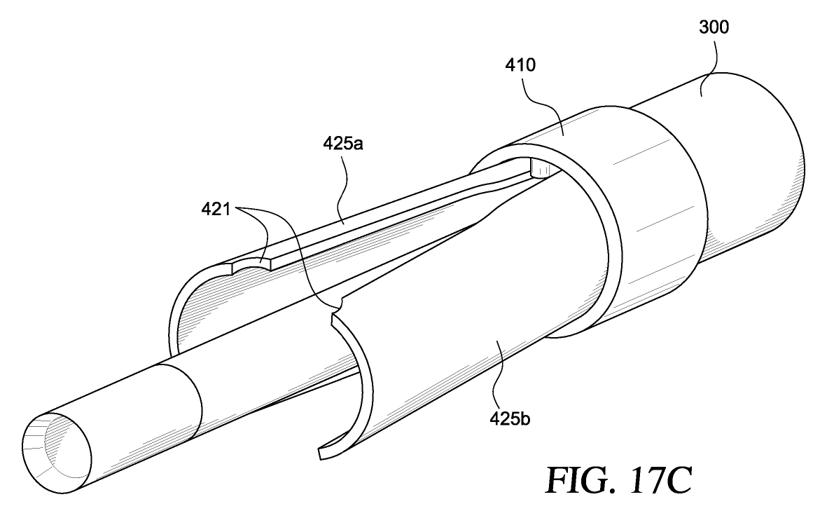

In further accordance with a preferred embodiment, a locking means provides an interface that prevents movement of the connector conduit 100 relative to the pushing element 300. Such locking means may include components that are integral with the pushing element 300, connector conduit 100, mounting element 200, and coring element 210. FIGS. 17A to 17C illustrate one embodiment of such a locking means. This embodiment combines a slot-and-key arrangement with a friction enhancing arrangement. The slot-and-key arrangement includes notch 421 (the slot) of pushing element 300 and holder button 430 (the key) of structural frame 101. Positioning holder button 430 into notch 421 prevents rotation of connector conduit 100 relative to pushing element 300 and prevents axial motion in one direction. Axial motion allowing removal of the connector conduit 100 from the applicator is not prevented in this embodiment. Rather, this axial motion is reduced by providing a friction enhancing arrangement consisting of squeeze ring 410 (which includes two groove pins 411) and squeeze arms 425a and 425b that cantilever from pushing element 300 to form wide groove 420a and narrow groove 420b. Alternatively, notch 421 could fit tightly around the circumference of holder button 430 to prevent movement of the connector conduit 100 relative to the pushing element 300 in both rotational and axial directions. As shown, notch 421 is divided, with one half cut from squeeze arm 425a and the other half from squeeze arm 425b. Alternatively, notch 421 could reside entirely within either squeeze arm. Alternatively, several notches 421 could be used.

When squeeze ring 410 is positioned at or near notch 421 as shown in FIG. 17B, squeeze ring 410 holds squeeze arms 425a and 425b tightly against connector conduit 100, creating a tight friction fit. In this position, groove pins 411 within wide groove 420a do not tend to separate squeeze arms 425a and 425b. When squeeze ring 410 is positioned as shown in FIG. 17C, groove pins 411 within narrow groove 420b tend to separate squeeze arm 425a and 425b to allow the connector conduit to be easily moved into position or removed. In a similar embodiment (not shown), the slot-and-key arrangement could include teeth (keys) that extend radially inwards from the inner diameter of squeeze arms 425a and 425b to fit into holder slots 431 of holder 130 of structural frame 101 (see FIG. 10A). In this embodiment, a squeeze ring (with groove pins) and squeeze arms similar to those shown in FIGS. 17A to 17C would be used to engage and disengage the teeth from holder slots 431, rather than to provide a tight friction fit.

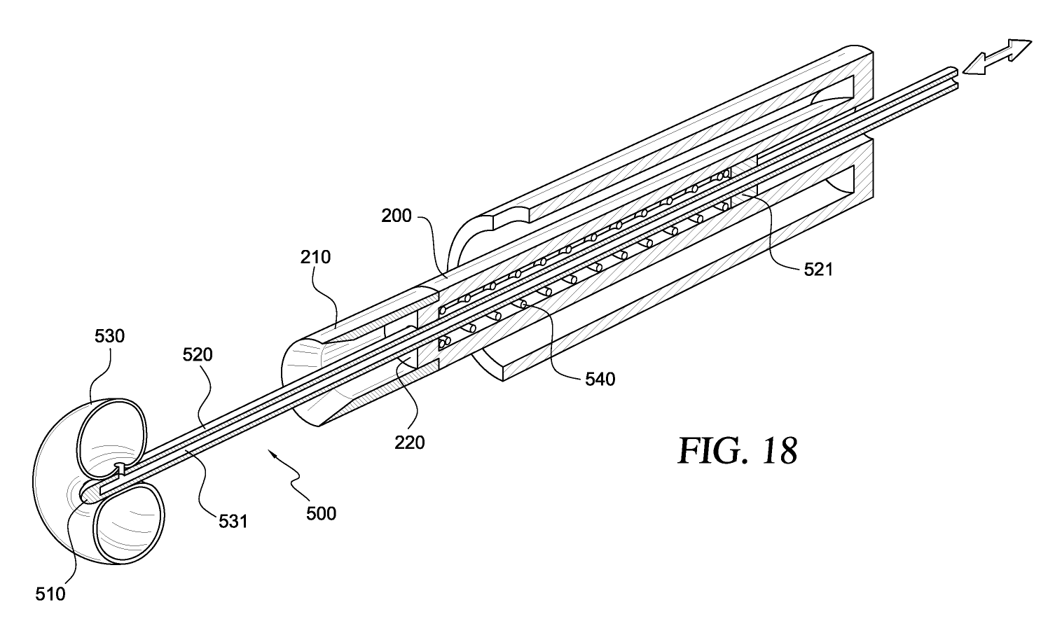

In accordance with a further embodiment of the present invention, a retractor component/element 500 with a generally tubular structure is located concentrically within the mounting element 200, as shown in FIG. 18. The retractor element 500 can slide axially relative to the mounting element 200. The retractor element 500 consists of a blunt tip 510, a tubular body 520, and an expanding element 530 that includes an access passage 531. The expanding element 530 is shown as a balloon in FIG. 18, which may be inflated and deflated with fluid (e.g., saline) through access passage 531 using a plunger and cylinder arrangement.

Retractor element 500 is held concentric within the mounting element 200 by centering plug 220 and sliding plug 521. Centering plug 220 is rigidly attached to mounting element 200, and sliding plug 521 is rigidly attached to tubular body 520. Since radial force from the heart wall tends to deflect the expanding element 530, tubular body 520 must have a sufficient stiffness to substantially resist such deflection. Such deflection may also be reduced by limiting the axial distance between the expanding element 530 and centering plug 220.

A coupling element, such as compression spring 540, slideably couples retractor element 500 to mounting element 200. Compression spring 540 biases refractor element proximally to ensure that expanding element 530 seats snugly against the inside wall of the ventricle to shape and partially flatten the ventricle wall (particularly at the apex) so that coring element 210 may cut perpendicular to the ventricle wall. Once the tissue plug is cut from the ventricle by coring element 210, spring 540 pulls the tissue plug fully within the coring element 210. In the preferred embodiment, expanding element 530 is a balloon in the shape of a circular torrid.

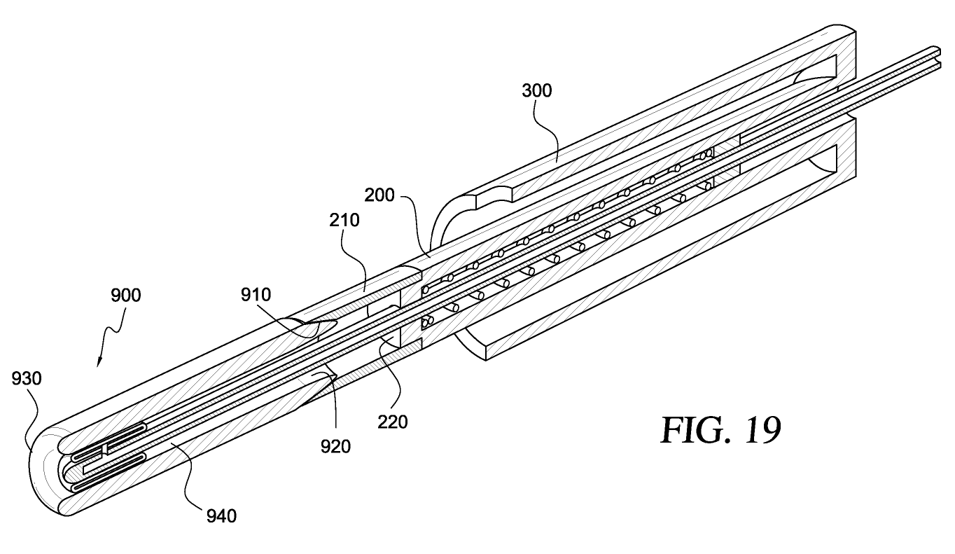

FIG. 19 illustrates a mounting and folding tool 900, which includes coring element taper 910, balloon taper 920, conduit taper 930, and retractor element port 940. Tool 900's outer diameter may be equal to or slightly larger than coring element 210's outer diameter to prevent damage to fabrics of the vascular graft 160 and outer fabric 161, when the connector conduit 100 is being mounted onto or demounted from mounting element 200. As an alternative, a thin-walled tube, such as a plastic shrink tube, may be positioned over outer diameters of tool 900 and coring element 210 to further prevent damage to fabrics slid past the sharpened edge 210a of the coring element. Coring element taper 910 fits snugly within coring element 210 to ensure a concentric fit between tool 900 and coring element 210, thereby further reducing the likelihood of damage to vascular graft 160 and outer fabric 161. Conduit taper 930 eases placement of vascular graft 160 onto tool 900. Tool 900 may be used to deflate and fold expanding element 530 by placing tool 900 onto retractor element 500 and by pushing and rotating (in one direction) tool 900 until coring element taper 910 contacts coring element 210. Balloon taper 920 provides a surface for controlled deflation and folding of the expanding element 530. Once the balloon is deflated and folded and the connector conduit 100 is fully mounted onto the applicator, tool 900 may be removed.

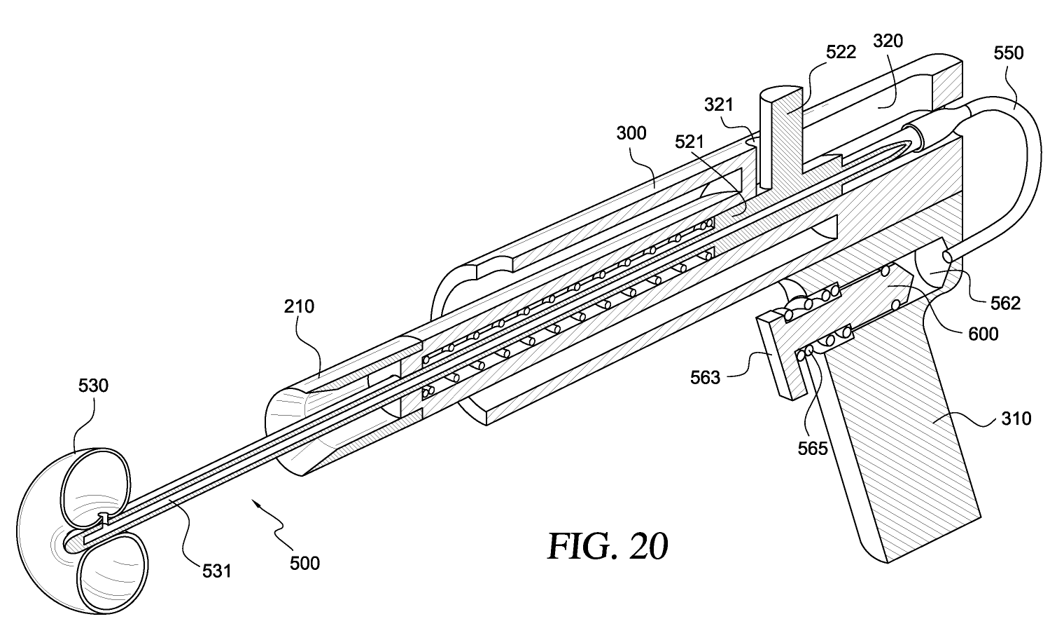

FIG. 20 illustrates an embodiment of an applicator assembly (connector conduit 100 not shown). In this assembly, the surgeon has independent control of the position of retractor element 500 and the volume of expanding element 530. Handle 310, which extends from pushing element 300 to form a pistol grip, provides a means for the surgeon to apply axial force and back-and-forth rotary motion while implanting connector conduit 100. The position of retractor element 500 is controlled by the position of retractor bolt 522 in slot 320 of pushing element 300. Retractor bolt 522 is rigidly attached to sliding plug 521 of retractor element 500. Slot 320 is extended circumferentially to form index 321, which may be used to hold the retractor element 500 fully extended (i.e., with expanding element 530 at maximum distance from coring element 210). Expanding element 530 is connected to cylinder 562 by access passage 531 and flexible tube 550. Expanding element 530 volume is controlled by the position of plunger 600 in cylinder 562. Cylinder 562 is oriented in handle 310 so that plunger 600 with trigger 563 forms a pistol handle with trigger arrangement. Expanding element 530 can be inflated with saline, when trigger 563 is squeezed. Plunger spring 565 may be used to deflate expanding element 530 when the trigger is released. Alternatively, trigger 563 could be replaced with a finger ring so that the user must apply force to control both inflation and deflation of expanding element 530, thereby eliminating the need for plunger spring 565. As a safety feature, the plunger 600 may include a latching device (not shown) that latches the plunger 600 with the balloon fully inflated, thereby preventing premature deflation of the balloon. A related safety feature may include another latching device (not shown) that latches plunger 600 with the balloon partially inflated, such as to prevent the tissue plug from coming off of retractor element 500. As one of many alternatives to handle 310, the handle could form a “T” with pushing element 300.

In operation, retractor bolt 522 is positioned in index 321 until the retractor element 500 is fully inserted into the ventricle and expanding element 530 is fully inflated. At that time, retractor bolt 522 is manually released from index 321, which allows compression spring 540 to retract retractor element 500 until expanding element 530 contacts the inside wall of the ventricle. A damping means (not shown) may be included to prevent sudden retraction of the retractor element 500 upon release from index 321. Also not shown is a safety latch or other means to prevent manual release of the retractor bolt 522 until the expanding element 530 is fully expanded. As the surgeon applies force and rotation using handle 310, compression spring 540 continues to displace retractor element 500. When retractor element 500 is fully retracted, expanding element 530 is also fully retracted to within coring element 210, indicating that the tissue plug has been successfully removed from the left ventricle and is within the coring element 210.

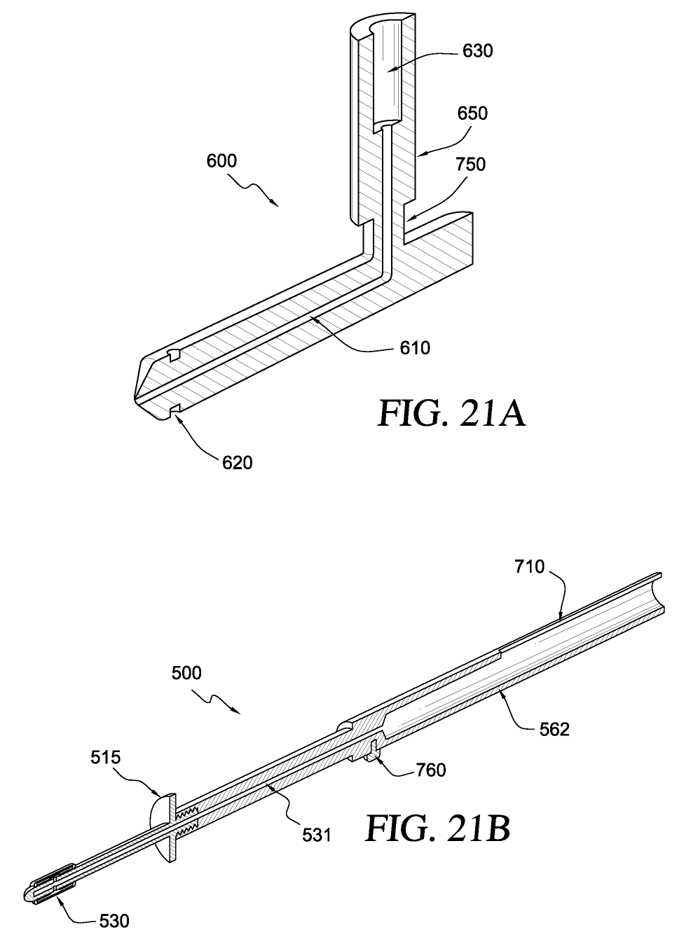

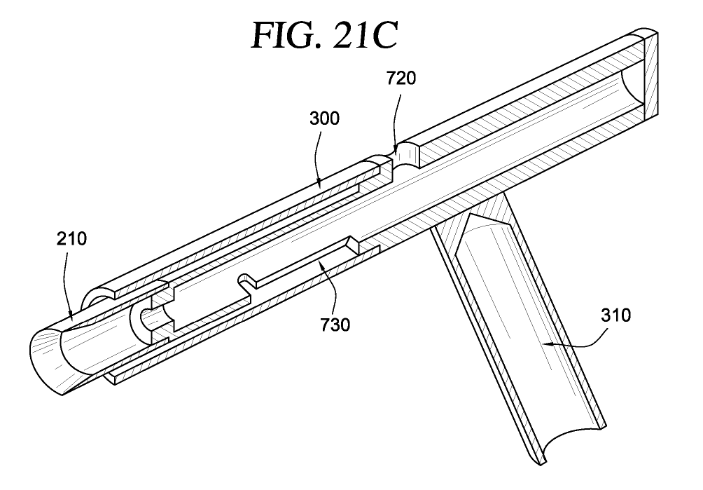

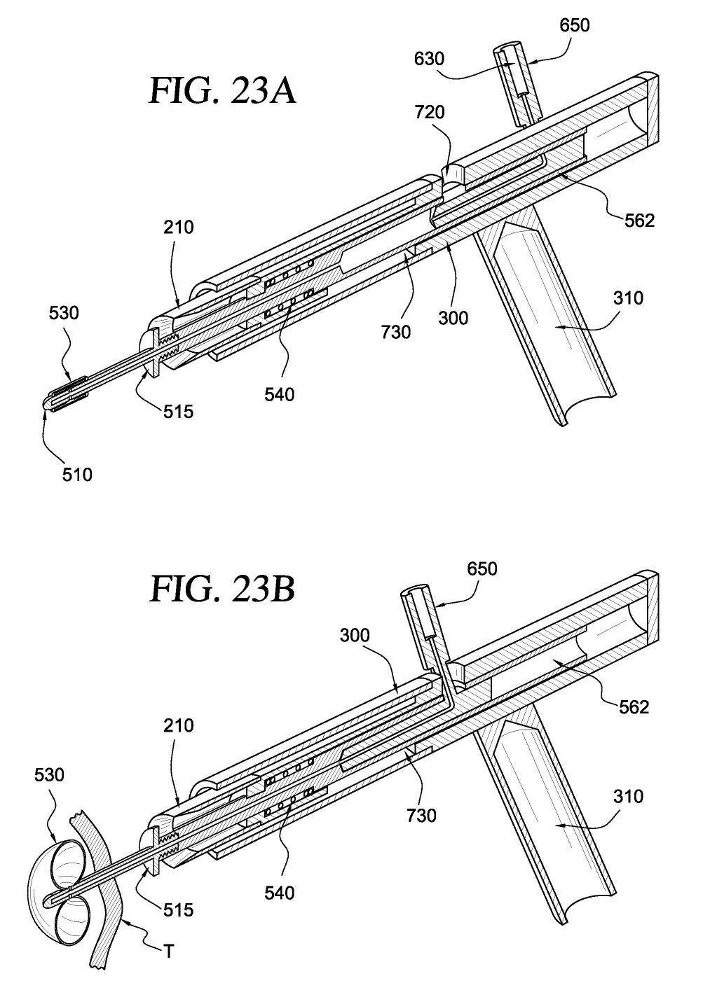

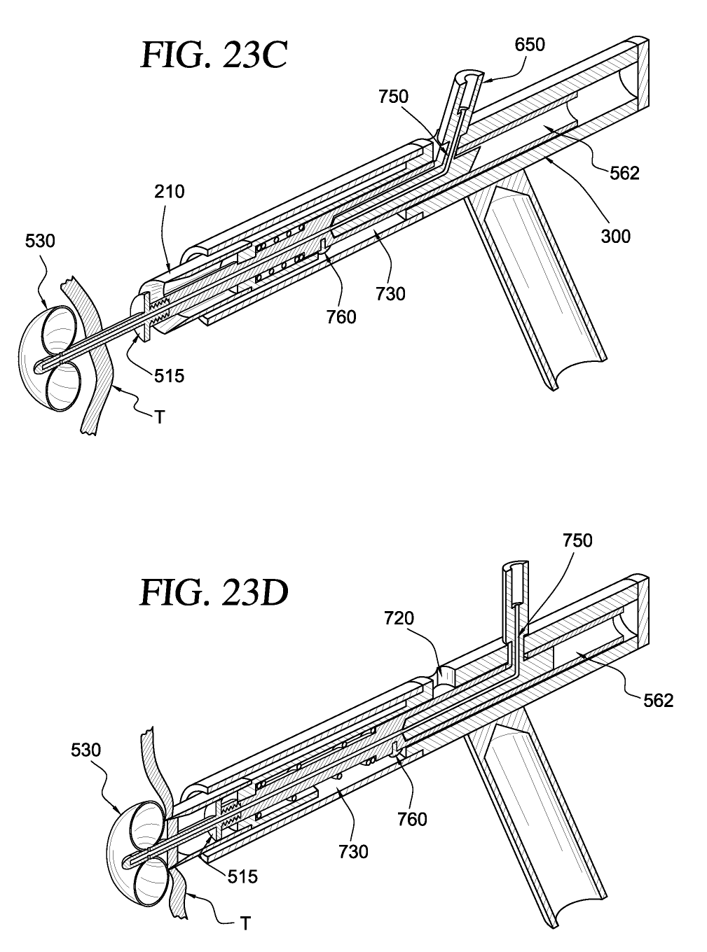

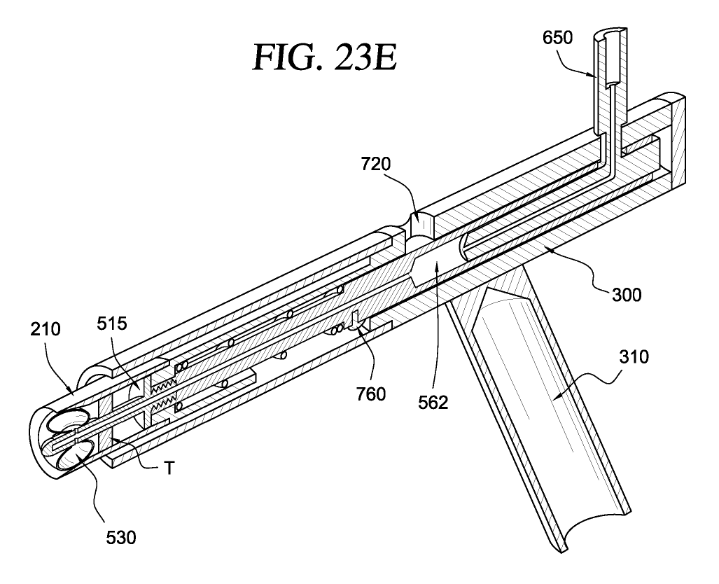

FIG. 21A to FIG. 21C are components of a preferred embodiment shown in FIGS. 23A-23E, that uses a sequencing element to coordinate the position of retractor element 500 with the expansion of expanding element 530 (FIG. 21B). In this embodiment, the sequencing element is a cam mechanism. The cam mechanism helps to ensure proper use of the applicator during implantation of connector conduit 100 (not shown). As shown in FIG. 21B, retractor element 500, referred to as the retractor assembly, includes cylinder portion 562 integrated therein. The retractor assembly is positioned concentrically within pushing element 300 during use. The retractor assembly contains elements of the cam mechanism formal therein, including cylinder cam slot 710, which is a slot cut completely through the cylinder 562 wall, and a retractor cam follower 760, which may be a pin or screw in cylinder 562 (as shown) or may be an integral part of cylinder 562. Retractor element 500 may include a section of increased diameter such as stopper disk 515 to prevent cutter element 210 from cutting the heart when retractor element 500 is initially inserted. FIG. 21A illustrates plunger 600 (in the form of a sequencing bolt as described below), which is positioned concentrically within cylinder 562 during use. Plunger 600 contains elements of the cam mechanism, including bolt portion 650 with plunger cam follower 750. Plunger cam follower 750 moves within cylinder cam slot 710 and pusher cam slot 720. Plunger 600 includes passage 610 and purge/fill valve 630 (valve body not shown). Valve 630 can be opened to allow fluid flow into and out of passage 610. When closed, valve 630 allows no fluid flow in either direction. Valve 630 may be connected (such as with a catheter) to a reservoir of saline, for example, to purge the expanding element 530, access passage 531 and any other volume in the flow circuit of air before filling these volumes with fluid (such as saline). O-ring groove 620 of plunger 600 contains an o-ring (not shown) to prevent loss of fluid.

FIG. 21C illustrates a positioning assembly, which is made up of rigidly connected components including pushing element 300, cutting element 210, and handle 310. The pusher assembly contains elements of the cam mechanism, including pusher cam slot 720 and retractor cam slot 730. The pusher cam slot 720 is a slot cut completely through the pushing element 300 wall to accommodate plunger cam follower 750.

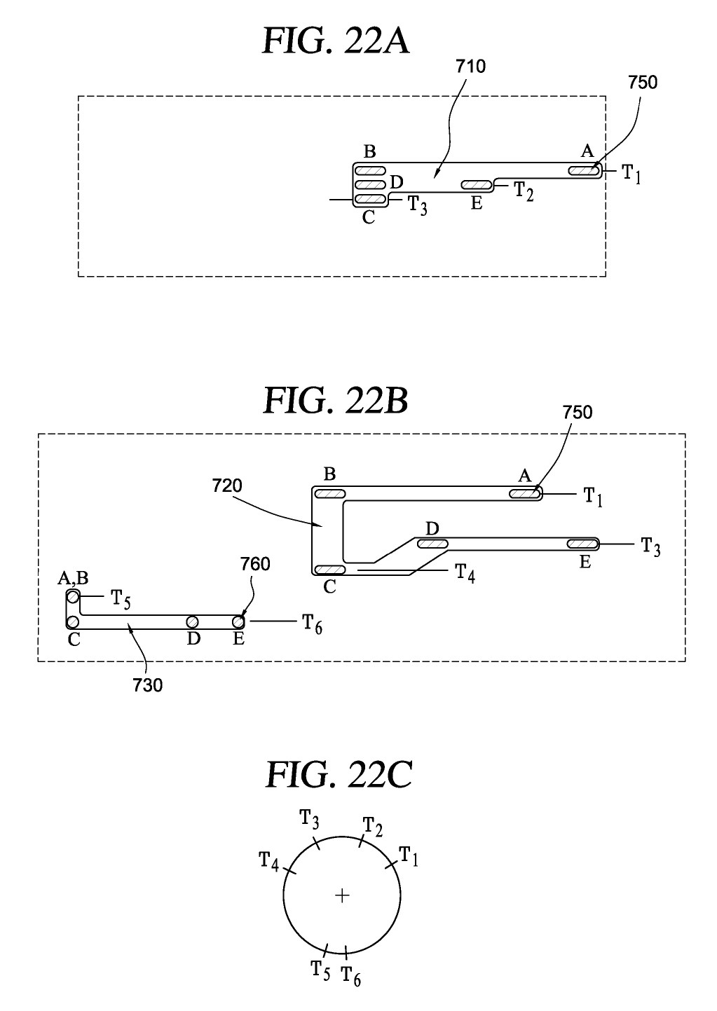

FIG. 22A to FIG. 22C illustrate operation of the cam mechanism. FIG. 22A illustrates cylinder cam slot 710 cut into cylinder 562 of FIG. 21B. Cylinder cam slot 710 contains three interconnected axial cam slots at angles θ₁, θ₂ and θ₃ around the circumference of cylinder 562, as further illustrated in FIG. 22C. The axial cam slot at each angle corresponds to a range of allowable axial positions of plunger 600 within cylinder 562. At angle θ₁, the axial length of the cam slot corresponds to the maximum stroke of plunger 600 within cylinder 562. This maximum stroke allows filling the expanding element 530 from minimum volume to maximum volume. At angle θ₂, the axial cam slot allows plunger 600 movement to provide expanding element 530 volumes ranging from maximum volume to an intermediate volume (at an intermediate stroke) that is greater than minimum volume but less than maximum volume. At angle θ₃, the axial cam slot retains plunger 600 at the position of maximum volume of the expanding element 530. FIG. 22A also illustrates positions A, B, C, D and E of plunger cam follower 750 within cylinder cam slot 710 during the steps of operation.

FIG. 22B illustrates pusher cam slot 720 and retractor cam slot 730 cut into the pusher assembly of FIG. 21C. FIG. 22B also illustrates positions A, B, C, D and E of plunger cam follower 750 within pusher cam slot 720 and retractor cam follower 760 within retractor cam slot 730 during the steps of operation. FIG. 22C illustrates angles θ₁ to θ₆ for cylinder 562 and the pusher assembly. For purposes of description, the value of the angles increases from θ₁ to θ₆. Pusher cam slot 720 includes angles θ₁ and θ₃, which may correspond with angles θ₁ and θ₃ of cylinder 562 (see FIG. 22A). Pusher cam slot 720 includes angle θ₄, which is larger than θ₃. The axial length of pusher cam slot 720 from position A to position B corresponds to the maximum stroke of the plunger 600, as described above. The axial length of pusher cam slot 720 from position C to position E corresponds to the intermediate stroke (as described above) plus the axial distance traversed by retractor cam follower 760 from position C to position E in retractor cam slot 730. Retractor cam slot 730 includes angles θ₅ and θ₆. Positions A and B at angle θ₅ prevent compression spring 540 from displacing cylinder 562 within the pusher assembly.

In operation, retractor cam slot 730 controls the motion of cylinder 562 within the pusher assembly. As shown in FIG. 22A and FIG. 22B, when plunger cam follower 750 (of sequencing bolt 600) is moved circumferentially from position B to position C in both cylinder cam slot 710 and pusher cam slot 720, retractor cam follower 760 is forced from position B to position C in retractor cam slot 730, which allows compression spring 540 (see FIG. 18) to push cylinder 562 axially within the pusher assembly. Retractor cam follower 760 within retractor cam slot 730 holds cylinder 562 at a constant angular position relative to the pusher assembly during movement from position C to positions D and E; therefore, movement of plunger cam follower 750 from position C to position D within pusher cam slot 720 forces cam follower 750 into the axial slot corresponding to angle θ₂ of cylinder 562.

Referring to FIGS. 23A to 23E, the applicator of the present invention is shown at various steps during use. Note that these figures do not include details of the locking means to securely hold the connector conduit 100. FIG. 23A to FIG. 23E correspond to positions A to E, respectively, which are described in FIG. 22A to FIG. 22C. Recognizing that individual surgeons may find alternative steps to properly use the invention, a representative sequence of steps for use of the applicator to implant a connector conduit is described. These steps include first preparing the applicator with the connector conduit. With the retractor assembly in the fully extended position as shown in FIG. 23A, a mounting and folding tool 900 is positioned into the coring element 210, as shown in FIG. 19. The connector conduit 100 of FIG. 14 is then loaded into the applicator by sliding connector conduit 100 over the folding tool 900 until sewing flange 170 contacts notch 421 (see FIG. 17). The connector conduit is then locked into place using the locking means. Tool 900 is then removed. A catheter is attached to purge/fill valve 630 and to a reservoir of saline. Valve 630 is opened. Sequencing bolt 600 is then moved back and forth from position A to position B several times to purge the fluid system of air and to fill the system with fluid, such as saline. Once the air is purged, sequencing bolt 600 is placed at position A, and tool 900 is again positioned into the coring element 210—this time to squeeze fluid from the balloon and to fold the balloon. When tool 900 is in place, valve 630 is closed, and the catheter is removed. Tool 900 is removed. The applicator with connector conduit is now ready for use, as shown in FIG. 23A.

Before implanting the connector conduit 100 into the ventricle wall, the portion of the prosthesis that includes the prosthetic valve or ventricular assist device, as examples, is connected to the aorta. This portion of the prosthesis also includes the female end of quick connect coupler 180. By implanting this portion of the prosthesis first, the time between insulting the heart by cutting a hole and beginning blood flow through the complete prosthesis is minimized.

A template with similar dimensions as connector conduit 100 is placed on the apex of the heart, and a marker is used to trace the circular outline of the connector onto the apex, in the planned location of insertion. Multiple (8 to 12) large pledgeted sutures (mattress sutures) of for example, 2-0 prolene, are placed in the apex surrounding the marked circle. With the connector conduit 100 loaded in the applicator of FIG. 23A, the sutures are brought through sewing flange 170 of the connector conduit 100. A knife is used to make a stab wound in the apex at the center of the circle. With the applicator in the position shown in FIG. 23 A, blunt tip 510 of retractor element 500 is inserted into the stab wound and pushed through the apex into the left ventricle chamber until stopper disk 515 contacts the epicardium (outside surface of the heart). Sequencing bolt 600 is moved from position A to position B to inflate the balloon behind tissue T of the heart wall (see FIG. 23B). The surgeon moves sequencing bolt 600 from position B to position C (see FIG. 23C) and then releases sequencing bolt 650. Beginning at position C of FIG. 23C, compression spring 540 pushes the retractor assembly from position C to position D (see FIG. 23D). When the retractor assembly moves from position C to position D, tissue T of the heart wall is first sandwiched between the balloon and the sharpened edge of the coring element 210a. By the surgeon using handle 310 to apply axial force and back-and-forth rotary motion, the sharpened edge of the coring element 210a cuts though the heart wall to form a plug of tissue T that resides in the coring element 210. At position D, the retractor assembly has been retracted until the balloon is in contact with coring element 210 and the tissue plug is fully within coring element 210. Also at position D, cylinder cam slot 710 has forced plunger cam follower 750 circumferentially to angle θ₂, thereby allowing deflation of the balloon to begin. Between position D (FIG. 23D) and position E (FIG. 23E), the balloon deflates to the intermediate volume (described earlier), and the retractor assembly retracts to its final position. If necessary, the surgeon may pull sequencing bolt 600 to its final position E.

Connector conduit 100 is now fully implanted. The sutures are tied, and hemostasis is checked. Additional sutures may be placed if needed. The locking means (not shown) holding the connector conduit in the applicator is released, and the applicator is partially removed to a position where a clamp can be placed directly on collapsible graft 160a to prevent blood flow through the conduit 160. Once the clamp is in place, the applicator may be completely removed from connector conduit 100. The male and female ends of quick connect coupler 180 may now be connected. Umbilical tape 187 may be tied around graft extension 186a to reduce any blood leakage, and stay sutures may be used to secure graft extension 186a to outer fabric 165. Once the flow passage of the prosthesis is purged of air, the clamp may be released to allow blood flow through the prosthesis. Flexible bend 140 is formed by pulling threads 143 and tying a knot. The connector conduit 100 is now fully implanted.

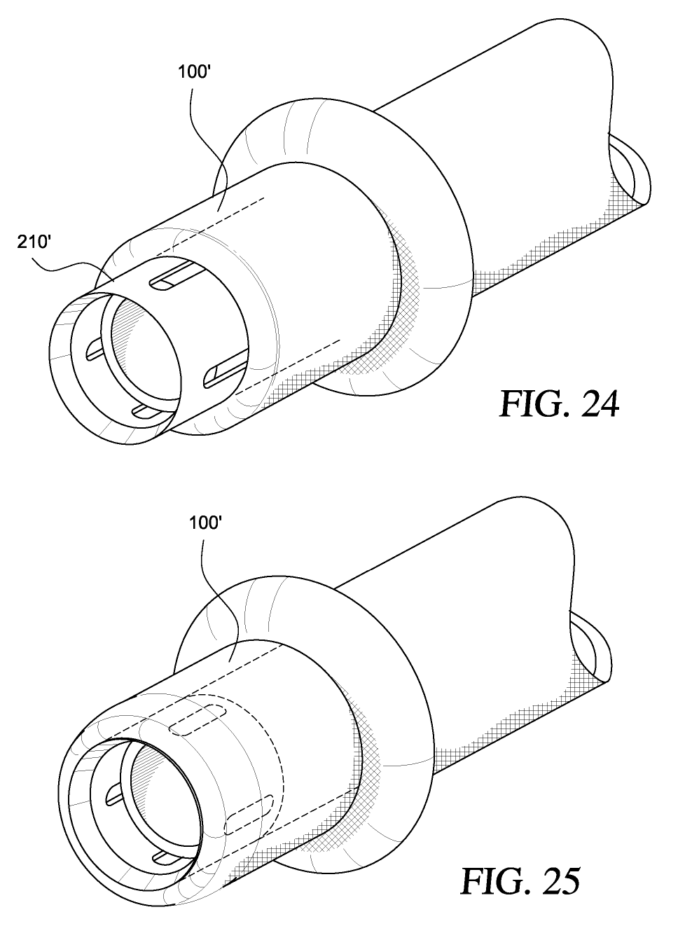

As illustrated in FIG. 24, an alternative embodiment, can use a connector conduit having an integral hole forming element. Hole forming element 210′ is integrally formed, i.e. formed as a single component, with respect to connector conduit 100′. Connector conduit 100′ can be loaded on an applicator (not having a separate hole forming element) in a manner similar to that disclosed above. After forming the hole and inserting the connector conduit into the hole, hole forming element 210′ can be withdrawn into a distal end of connector conduit 100′, as illustrated in FIG. 25, to reduce the possibility of unintended tissue damage. Such withdrawal can be accomplished by the sequencing means, a manual mechanism on the applicator, or with a separate instrument.

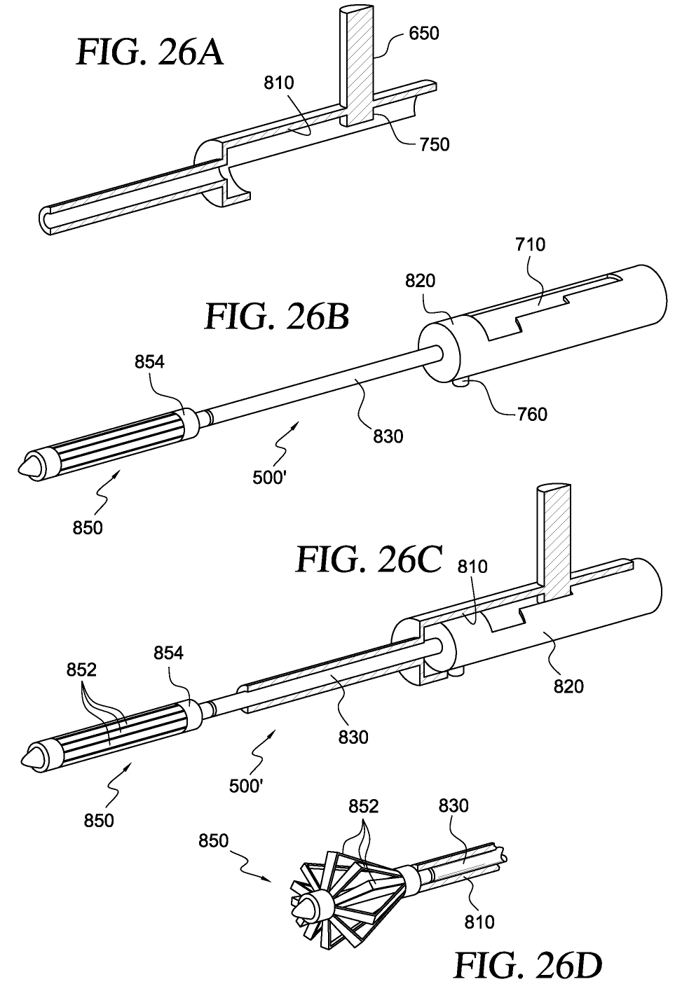

In the preferred embodiment described above, the expansion element is a balloon. However, an alternative expansion element, in the form of an umbrella mechanism, is illustrated in FIGS. 26A-26D. Retractor 500′ includes cylinder 810 (shown in cross section), and piston element 820 slideably disposed in cylinder 810. Bolt 650 having follower 750 is formed on cylinder 810. Shaft 830 extends from piston element 820 and has umbrella mechanism 850 formed on an end thereof. Umbrella mechanism 85 included plural bendable leaf elements 852 that are fixed to shaft 830 at the end of shaft 830. Leaf elements 852 are fixed to ring 854 at the other end thereof. Ring 854 is slideably disposed on shaft 830. Accordingly, movement of shaft 830 to the right in the FIGS. causes ring 854 to be pushed toward the end of shaft 830 as ring 854 abuts an end of cylinder 810, as shown in FIG. 26 D. Slot 710 guides follower 750, and bolt 650 cooperates with remaining elements in the sequencing mechanism in the manner described above, to coordinate the expansion state of expansion element 850.

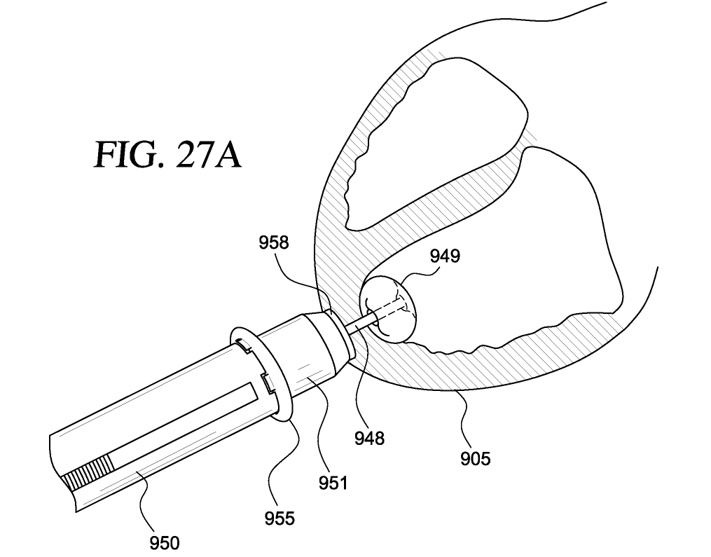

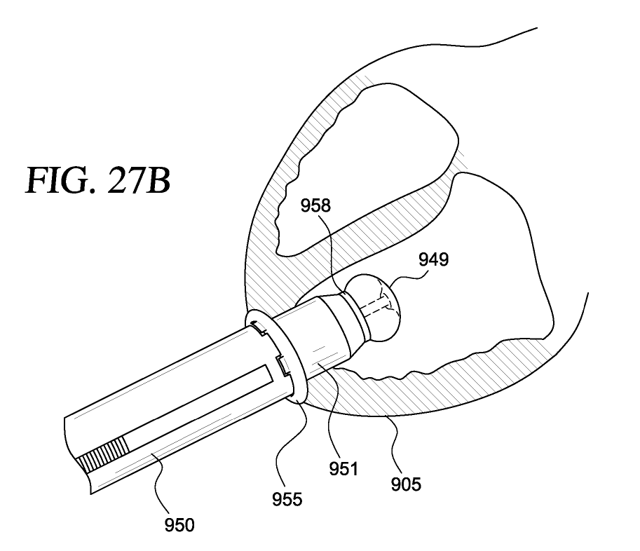

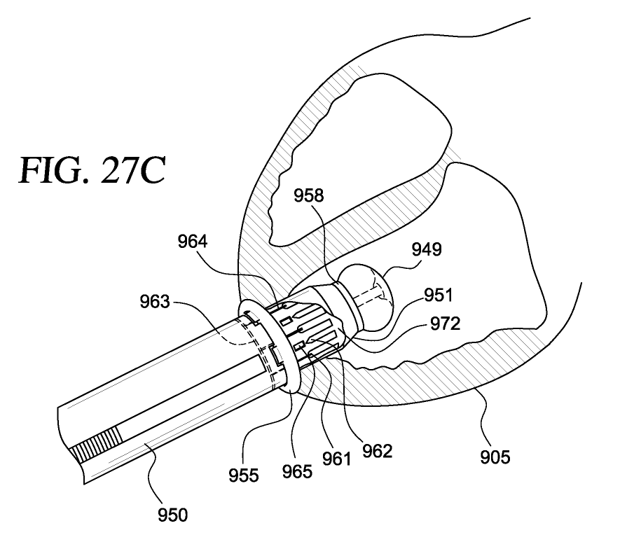

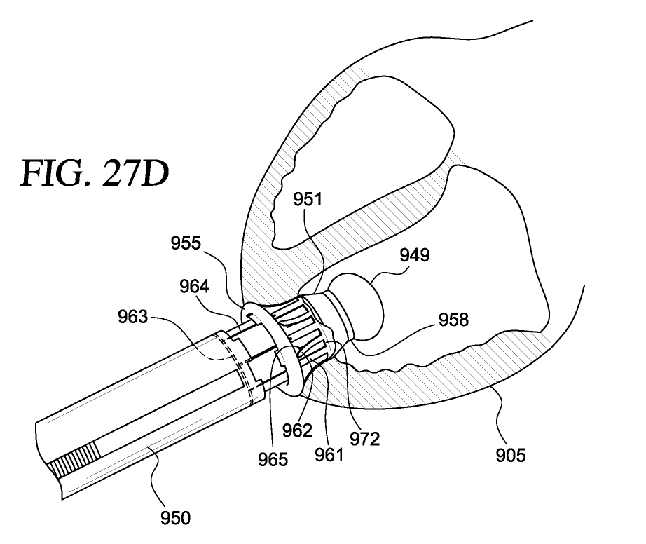

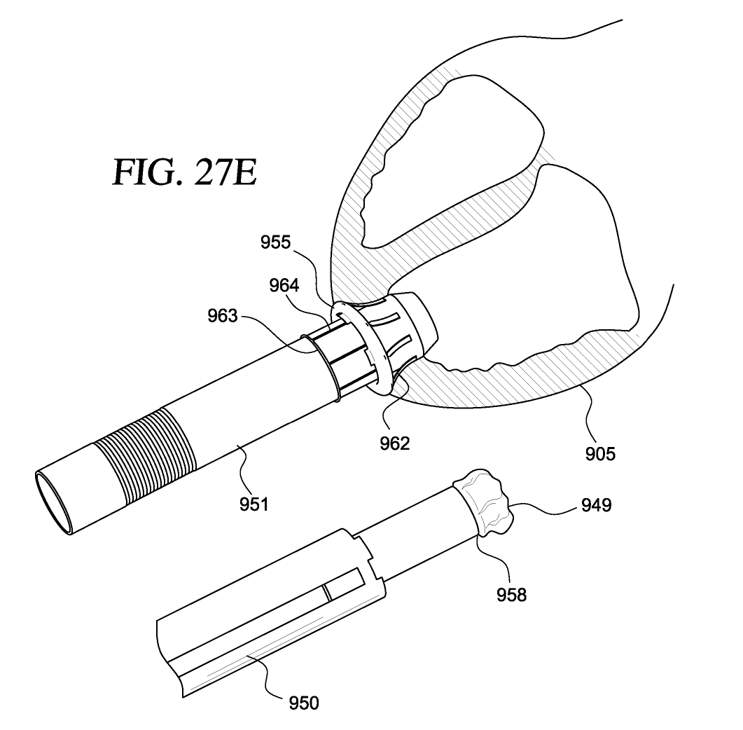

As illustrated in FIGS. 27-29, the invention also relates to a connector conduit with applicator that eliminates the need to sew the connector conduit to the apex. This apparatus of the invention generally includes a connector conduit operable to be inserted through a hole in a wall of the organ, a flange element positioned on the connector conduit adapted to prevent over-insertion of the connector conduit, and a retention means positioned on the connector conduit. The retention means is preferably adapted to be engaged with the wall of the organ to prevent movement of the connector conduit relative to the wall of the organ after the connector conduit is inserted through the hole in the wall of the organ.

As described in the embodiments illustrated in FIGS. 1-26 above, during operation, the tip of a retractor element 948 is pushed through the wall of an organ 905. An expansion element 949, such as a balloon element, is attached to retractor element 948 near the tip. As the tip of retractor element 948 is pushed through the wall of organ 905, expansion element 949 is also pushed through the wall of organ 905. After expansion element 949 is positioned within organ 905, expansion element 949 is expanded from a compressed or deflated state to an expanded or inflated state, and retractor element 948 is withdrawn from the organ 905 until expansion element 949 is adjacent to the inner surface of the wall of organ 905, and coring element 958 is adjacent to the outer surface of the wall of organ 905. At this point, coring element 958 is used to form a hole in the wall of organ 905, the resulting tissue plug is removed, and connector conduit 951 is push through the hole in organ 905 until the leading edge of a flange element 955 (previously referred to as the sewing flange 170) is adjacent to the outer surface of organ 905.

Generally, during operation, the connector conduit is inserted through the hole in the wall of the organ until the flange element comes into contact with the wall of the organ, and the retention means is engaged with the wall of the organ after the connector conduit is inserted through the hole in the wall of the organ. The retention means is operative to prevent movement of the connector conduit relative to the hole in the wall of the organ after insertion of the connector conduit into the organ. In particular, the retention means generally prevents the force resulting from blood pressure within the organ (i.e. within the ventricle if the organ is a heart) from pushing the connector conduit out of the hole in the wall of the organ.

FIGS. 27A-27E illustrate an embodiment of the invention in which the retention means consists of a plurality of retaining pins 962. The retaining pins 962 improve homeostasis by providing a squeezing force to press the heart wall against connector conduit 951. Retaining pins 962 are preferably positioned circumferentially around the connector conduit, such that they are inserted into the hole in the wall of the organ when the connector conduit is inserted through the hole in the wall of the organ. In addition, the retaining pins are preferably maintained in a passive state adjacent to an outer surface of the connector conduit until entering into engagement with the wall of the organ.

In particular, the plurality of retaining pins 962 are connected to a ring 972, which is positioned circumferentially around connector conduit 951, and contained below the surface of connector conduit 951 (See FIGS. 27C-27D). A plurality of tabs 961 are also connected to ring 972 in a similar manner to retaining pins 962. Both retaining pins 962 and tabs 961 extend axially along connector conduit 962 as is shown in FIGS. 27C-27E. Retaining pins 962 preferably have a sharpened tip.

In addition, a means for causing the retaining pins to engage the wall of the organ to prevent movement of the connector conduit relative to the wall of the organ may also be used. The means for causing the retaining pins to engage the wall of the organ to prevent movement of the connector conduit relative to the wall of the organ may comprise a plurality of skid elements and pull wires, for example. For example, a plurality of pull wires 964 are connected to the ends of tabs 961, such that when an axial force is applied to pull wires 964, pull wires 964, retaining pins 962, and ring 972 are all subjected to the same force, and can move axially upon application of sufficient force. Each pull wire 964 is attached to a pull ring 963, which provides a means to apply an equal pulling force to each pull wire simultaneously. Pull wire 963 is preferably connected to applicator 950 such that extraction of applicator 950 results in application of an axial force on pull ring 963, and, accordingly, on retaining pins 962.

Furthermore, as is shown in FIG. 27C, connector conduit 951 is slightly modified in this embodiment to include a plurality of skids 965, which are positioned circumferentially around connector conduit 951 in such a way that each retaining pins 962 is preferably positioned in axial alignment with at least one skid 965. Each skid 965 comprises a sloping or curved surface that extends tangentially upwards from the axial plane of the connector conduit 951 in which the retaining pins 962 are positioned towards the outer surface of connector conduit 951.

During operation, when applicator 950 is extracted after installation of connector conduit 951, an axial force is applied to pull ring 963, and pull ring 963 slides axially along connector conduit 951 away from organ 905. As pull ring 963 moves along connector conduit 951, pull wires 964 exert a force on tabs 961, causing ring 972, and retaining pins 962, to slide axially along connector conduit 951 as well. As is illustrated in FIG. 27D, as retaining pins 962 slide along connector conduit 951, the tips of retaining pins 962 come into contact with skids 965, and are guided along the curved or angled surface of skids 965. As movement of retaining pins 962 continues, the tips of retaining pins 962 pierce the outer surface of connector conduit 951 and the wall of organ 905 (See FIG. 27E). Axial movement of pull ring 963 and retaining pins 962 continues until retaining pins 962 come into contact with flange element 955, at which point pull ring 963 disengages from applicator 950. The barb-like connection between retaining pins 962 and the wall of organ 905 prevent disengagement of connector conduit 951 from organ 905 without the use of additional sutures. In addition, the engagement of the retaining pins with the wall of the organ radially squeezes the wall of the organ against the connector conduit, thereby preventing any leakage of blood or fluids from within the organ around the engagement of the wall of the organ and the connector conduit.

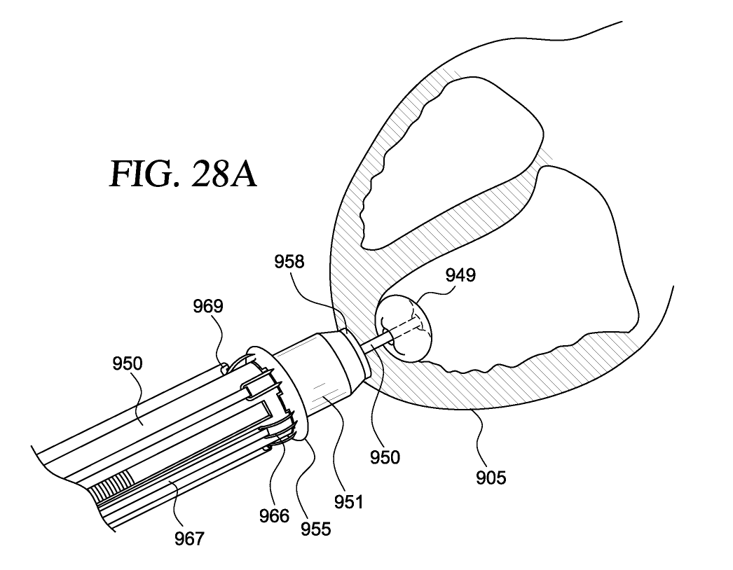

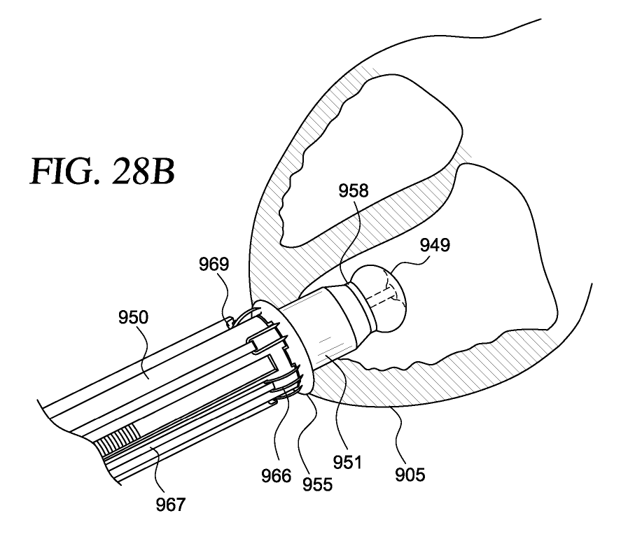

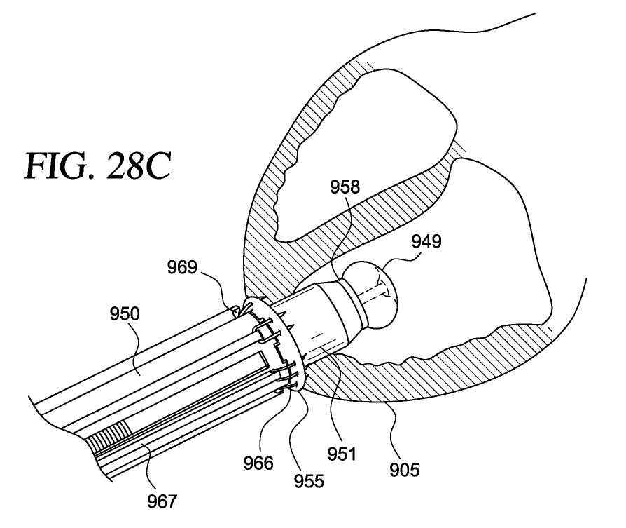

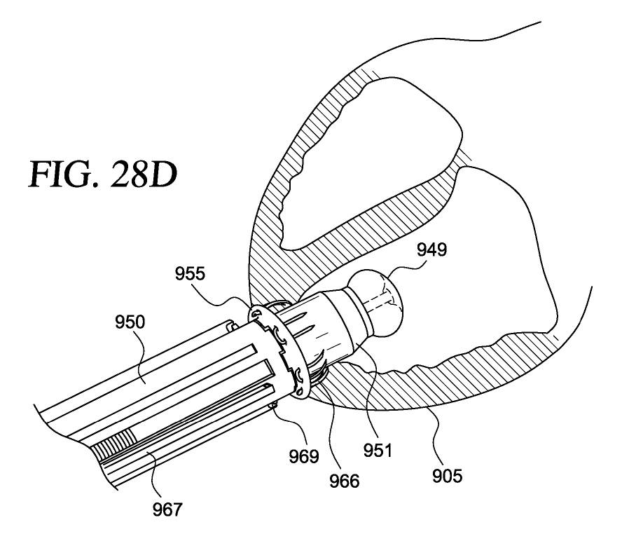

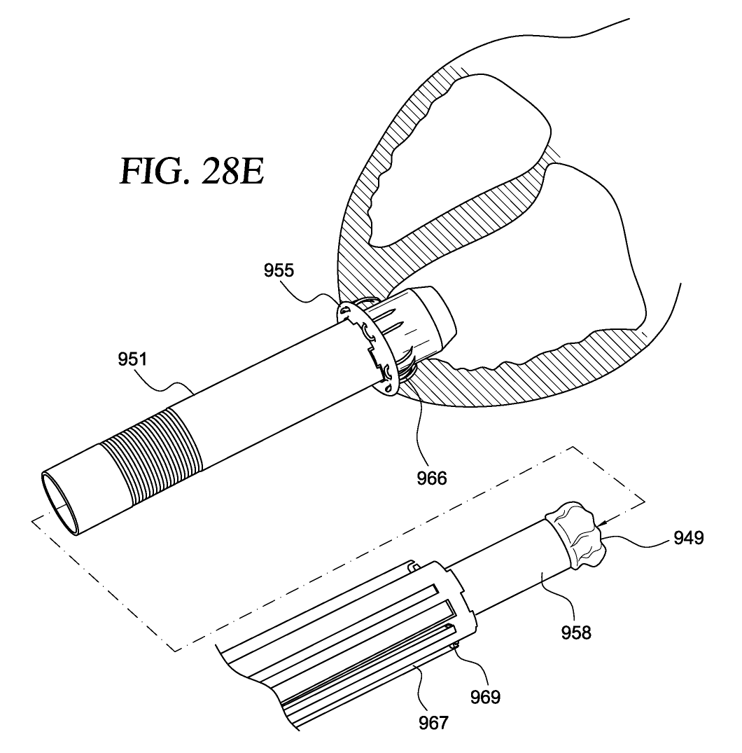

FIGS. 28A-28E illustrate an embodiment of the invention in which a plurality of prongs 966 are used as the retention means to prevent dislodgement of the connector conduit 951 from the wall of organ 905. The prongs are preferably positioned circumferentially around the connector conduit such that the prongs, when in an initial passive state, are positioned outside of the organ after the connector conduit has been inserted through the hole in the wall of the organ. Prongs 966 may also improve homeostasis by providing a squeezing force to press the heart wall against connector conduit 951.

Prongs 966, which are preferably shaped like curved staples, are positioned around the surface of applicator 950 in such a manner that the tips of each prong 966 is generally adjacent to the outer surface of flange element 955. After connector conduit 951 is inserted through the wall of organ 905 (as is shown in FIG. 28B), prongs 966 are inserted through flange element 955 and the outer surface of organ 905 (see FIG. 28C-28D), thereby securing connector conduit 951 to organ 905. Thus, it is clear that, after the connector conduit has been inserted through the hole in the wall of the organ, the prongs are adapted to be inserted through a plurality of holes in the flange element into the wall of the organ, thereby entering into engagement with the wall of the organ.

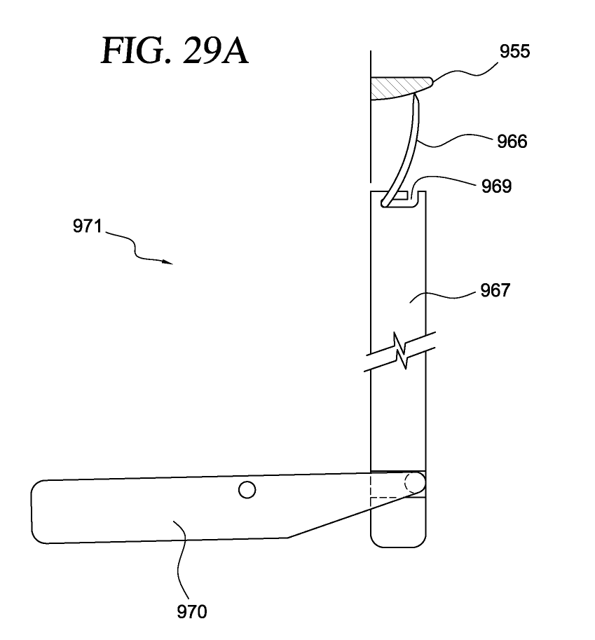

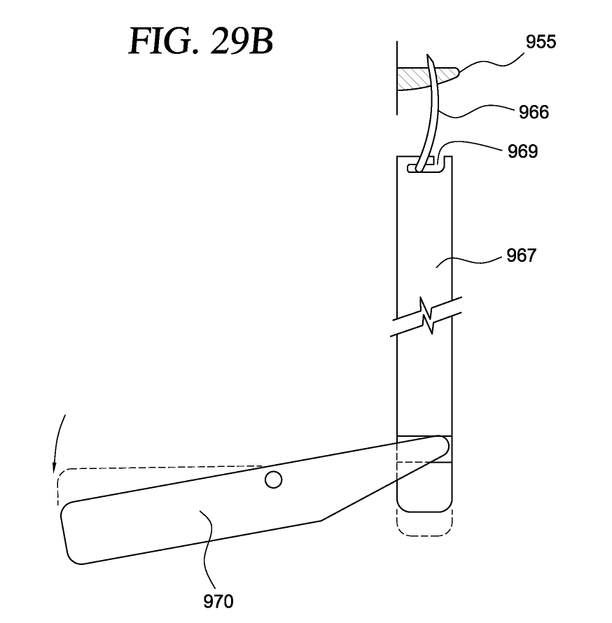

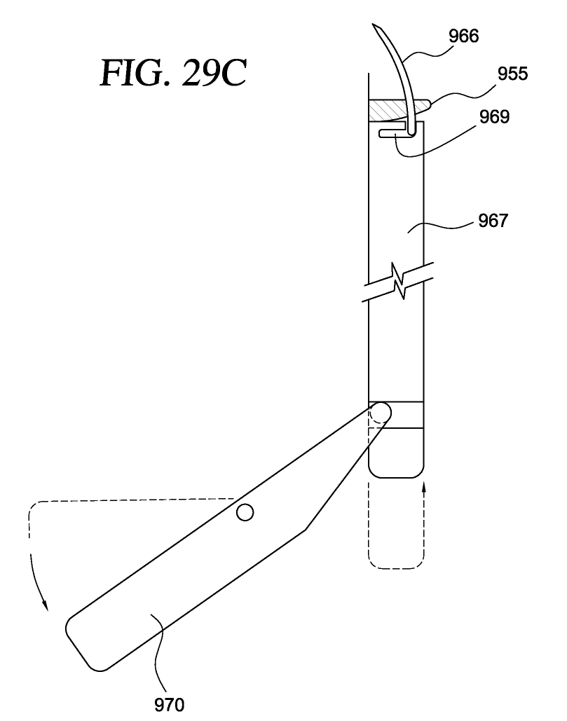

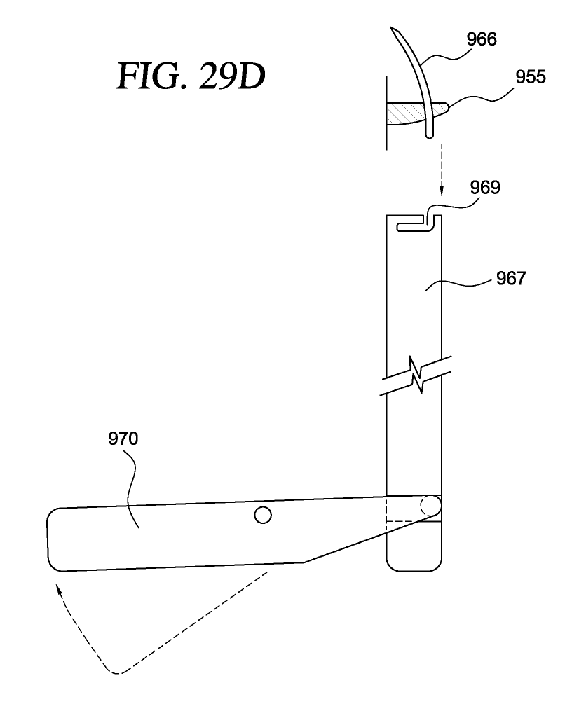

A prong installation element may be used which is adapted to insert the prongs through the holes in the flange element into the wall of the organ, thereby causing the prongs to enter into engagement with the wall of the organ. For example, the axial force needed to insert prongs 966 through flange element 955 and into organ 905 may be provided by a plurality of one or more prong deployment mechanisms 971. Each prong deployment mechanism 971, illustrated in FIGS. 29A-29D, generally comprises two components including a prong installation lever 970 and a prong installation element 967. Each prong installation element 967 is connected to a prong installation lever 970 with a hinge such that movement of a prong installation lever 970 results in movement of the corresponding prong installation element 967. Each prong installation element 967 extends longitudinally along surface of applicator 950. As is illustrated in FIGS. 29A-29D, each prong installation element 967 includes a curved slot 969 near one end, which serves as a deployment means for a prong 966.

In the preferred embodiment illustrated in FIG. 28, a plurality of prong installation elements 967 are arranges in a radial fashion around applicator 950. Any number of prongs may be used, for example, six or eight prongs. Most preferably, there are equal numbers of prong installation elements 967 and prongs 966. The installation and application of an exemplary prong will now be described with reference to the FIGS. 28-29. During operation, prong 966 is predisposed within slot 969 of prong installation element 967, with the tips of prong 966 being positioned generally adjacent to flange element 955. As a force is applied to prong installation lever 970, prong installation element 967 slides axially along applicator 950 towards flange element 955. Because prong 966 is positioned within slot 969, the movement of prong installation element 967 along applicator 950 results in axial movement of prong 966 as well. The tips of prong 966 are pressed through flange element 955 and into the wall of organ 905. Slot 969 is designed such that the curved characteristics of prong 966 result in prong 966 sliding out of slot 969 as prong 966 is pressed further and further through flange element 955 and into organ 905. Thus, when prong 966 has been fully inserted through flange element 955, prong 966 will no longer be positioned within slot 969. At this point, applicator 950 and prong deployment mechanism 971 may be removed from connector conduit 951, and prong 966 will remain inserted through flange element 955 and within the wall of organ 905. The curved connection between prong 966 and the wall of organ 905 prevent disengagement of connector conduit 951 from organ 905 without the use of additional sutures. In addition, the engagement of the prongs with the wall of the organ radially squeezes the wall of the organ against the connector conduit, thereby preventing any leakage of blood or fluids from within the organ around the engagement of the wall of the organ and the connector conduit.

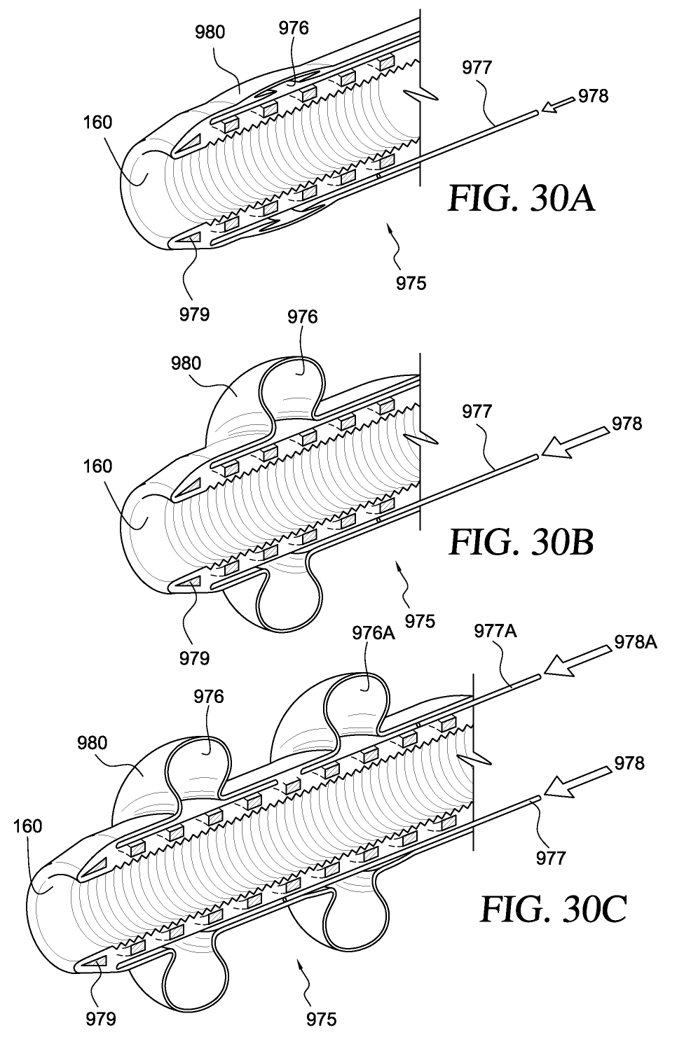

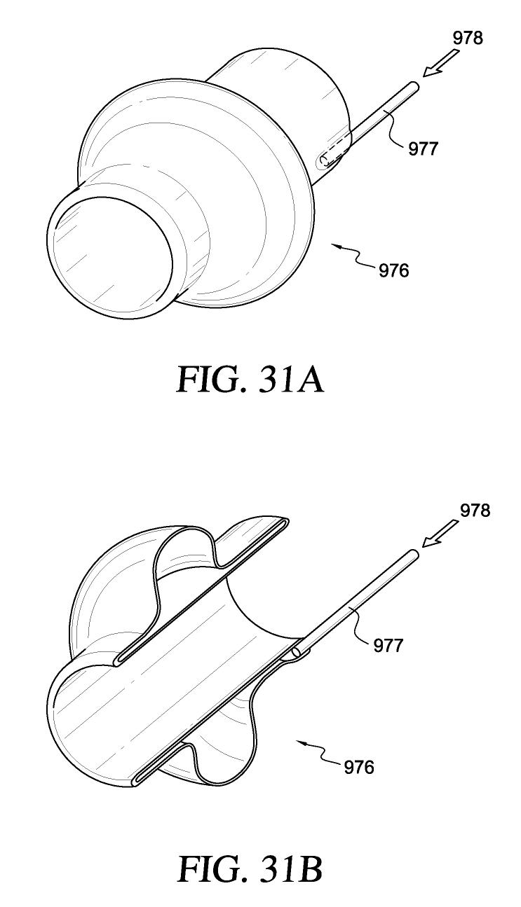

FIGS. 30A-30B illustrate an embodiment of the invention wherein a balloon 976 is used as the retention means to retain connector conduit 975 securely within the organ. Balloon 976 should be positioned on the connector conduit, such that the balloon is inserted through the hole in the wall of the organ as the connector conduit is inserted through the hole in the wall of the organ. FIGS. 31A-31B provide a more detailed view of balloon 976.

During operation, connector conduit 975 is inserted through the wall of the organ as is described above with balloon 976 preferably being in a initial deflated state until after the balloon and the connector conduit are inserted through the hole in the wall of the organ. (FIG. 30A). After insertion of the connector conduit through the wall of the organ, with balloon 976 residing within the organ, balloon 976 is inflated from the initial deflated state to an expanded state to prevent the pressure in the organ from pushing connector conduit 975 out, and to enter into engagement with the wall of the organ and preventing movement of the connector conduit relative to the hole in the wall of the organ. (FIG. 30B). In addition, the engagement of the inflated balloon with the wall of the organ axially squeezes the wall of the organ between the balloon and the flange element, thereby preventing any leakage of blood or fluids from within the organ around the engagement of the wall of the organ and the connector conduit. A coring knife may be used as a hole forming element to cut a hole in the organ through which connector conduit 975 is inserted.

Balloon 976, which may be formed of any suitable materials including, for example, polyurethane or polyethylene terephthalate (PET, polyester), is packaged in a deflated state (FIG. 30A) between an outer fabric sleeve 980 and a stent 979 of the connector conduit 975. Outer fabric sleeve 980 and vascular graft 160 are connected by any suitable connection means, for example, sutures.

As is shown in FIG. 31A-31B, balloon 976 is preferably formed from a single piece of material, such as a generally cylindrical sleeve, to minimize the possibility of leakage. The cylindrical sleeve may be folded back on itself longitudinally and fused to form balloon 975 as it is shown in the figures. In particular, the cylindrical sleeve may have a substantially constant diameter except for the portion of the sleeve that will be used for the expanding portion of the balloon. This portion should have a larger diameter to allow for the expansion of the balloon when inflating from the initial deflated state to the inflated state. In addition, the diameter of the remaining portions of the sleeve should not significantly change in response to the inflation of the balloon portion of the sleeve because the inner and outer portions of the sleeve are sandwiched between the outer fabric sleeve 980 and stent 979.

After connector conduit 975 is inserted through the wall of the organ, balloon 976 is inflated using a suitable biocompatible material provided by a fill tube 977. FIGS. 31A-31B show balloon 976 in its expanded state. It is preferred that balloon 976 be filled to a predetermined pressure (for example, 15 psi), thereby allowing the balloon to inflate and tightly engage the wall of the organ. In this manner, the inflation of the balloon consistently creates a tight seal against the wall of the organ regardless of variation in the shape of the organ or the thickness of the wall of the organ. In particular, since the degree of inflation is preferably based on the pressure within the balloon, different balloons will be inflated to different volumes until the predetermined pressure is reached.

Directional arrow 978 indicates the direction of flow for the biocompatible material during inflation of balloon 976. As balloon 976 expands, the shape of outer fabric sleeve 980 conforms with the expanding shape of balloon 976. Thus, it is preferred that outer fabric sleeve 980 be formed into a shape that allows for the expansion of balloon 976 without any significant deformation or stretching. To facilitate this, outer fabric sleeve 980 may have folds or the like prior to expansion of balloon 976. A removable sheath (not shown) may also be placed over outer fabric sleeve 980 while the connector is inserted into the heart wall to reduce any effects of the folded outer fabric. An exemplary material for outer fabric sleeve 980 is Dacron.

Balloon 976 may be filled with any suitable biocompatible material. Examples of suitable biocompatible materials include saline, or a silicone or polyurethane foam that is injected as a polymer and solvent which solidifies into a sponge-like permanent implant. An example of use of such a material is described in U.S. Pat. No. 6,098,629, which describes an endoscopic procedure to treat GERD (gastro esophageal reflux disease) by injecting this liquid polymer directly into the lower esophageal sphincter using a needle catheter. In the case of saline, it is possible that the saline could leak out of the balloon over time. Since the implant is grown in or chronic after about 8 weeks (i.e. the tissue has grown into the outer fabric sleeve), the saline would have to reliably remain in the balloon for at least that amount of time. Once the implant is chronic, it can only be removed by cutting it out, so the saline-filled balloon is not needed. Also, since the balloon is completely enclosed between the outer fabric sleeve and the inner portion of the connector conduit, a failed balloon after the implant is chronic cannot escape to cause problems, such as an embolus.

In an alternative embodiment shown in FIG. 30C, connector conduit may include a plurality of balloons 976 and 976A. For example, balloon 976 could reside inside the organ and balloon 976A could reside outside the organ, where the suture ring has been located for the other embodiments described above. When two balloons are used as expansion elements in this configuration, the balloons effectively compress the wall of the organ, thereby securing the connector conduit to the organ. It should be noted that it is preferred that a flange element still be used in this configuration to prevent over-insertion of the connector conduit into the organ. In this arrangement, the flange element can be positioned on the pusher assembly of the applicator instead of on the connector conduit. As such, the second balloon 976A could be positioned immediately adjacent to the flange element. Each balloon used in this manner preferably has its own fill tube to prevent migration of saline out of the organ. For example, in FIG. 30C, balloon 976 is inflated via fill tube 977, and balloon 976A is inflated via fill tube 977A.

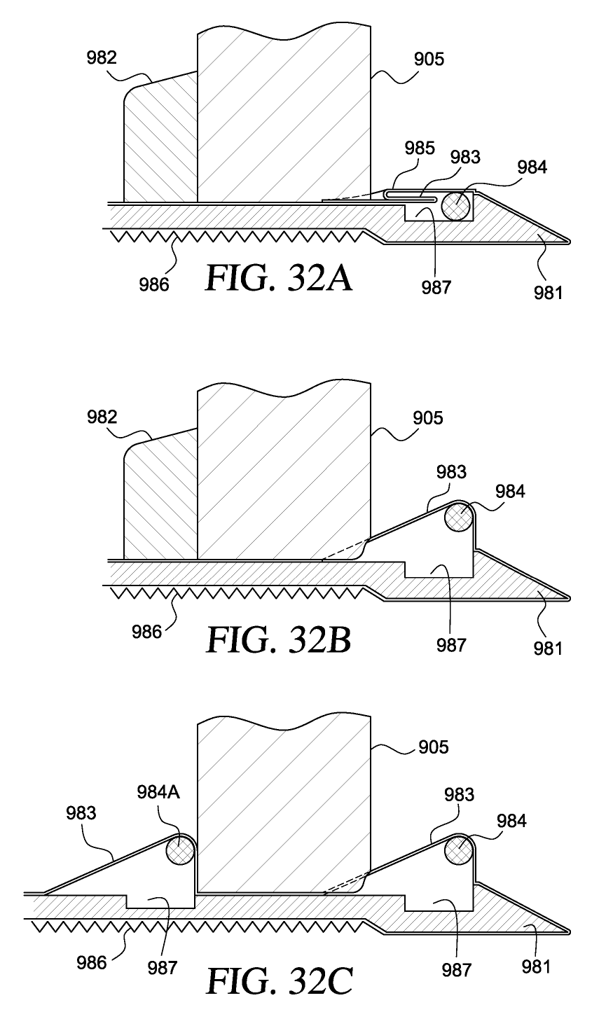

FIGS. 32A-32B illustrate an embodiment of the invention wherein a torsion spring is used as the retention means to retain the connector conduit securely within the organ. In particular, the torsion spring is preferably positioned on the connector conduit such that the torsion spring, when in an initial compressed state, is inserted through the hole in the wall of the organ as the connector conduit is inserted through the hole in the wall of the organ.

As is shown in the figures, a stent 981 is attached to a vascular graft 986 for insertion through the wall 905 of the organ. A torsion spring 984 is positioned, in a compressed state, in a circumferential groove 987 around stent 981. Vascular graft 986 extends around the tip of stent 981 and is connected to an outer fabric sleeve 983 by any suitable means, for example, sutures. Outer fabric sleeve 983 also covers torsion spring 984, and torsion spring 984 is preferably retained in a compressed state by a sheath 985 which is positioned over torsion spring 984 and outer fabric sleeve 983. FIG. 32A shows torsion spring 984 in a compressed state.

During operation, the connector conduit is inserted through the wall of the organ until flange element 982 contacts the outer surface of wall 905, as is described above. After being properly positioned, with torsion spring 984 residing inside the organ, sheath 985 is withdrawn through the wall of the organ and torsion spring 984 is allowed to expand from the initial compressed state to an expanded state, thereby entering into engagement with the wall of the organ and preventing movement of the connector conduit relative to the wall of the organ. In addition, the engagement of the expanded torsion spring with the wall of the organ axially squeezes the wall of the organ between the torsion spring and the flange element, thereby preventing any leakage of blood or fluids from within the organ around the engagement of the wall of the organ and the connector conduit.

As torsion spring 984 expands, the shape of outer fabric sleeve 983 conforms with the expanding shape of torsion spring 984. Thus, it is preferred that outer fabric sleeve 983 be formed into a shape that allows for the expansion of torsion spring 984 without any significant deformation or stretching. To facilitate this, outer fabric sleeve 983 may have folds or the like prior to expansion of torsion spring 984. FIG. 32B shows an exemplary shape of outer fabric 983 when torsion spring 984 is in an expanded state.

As described above with reference to balloons, the connector conduit may also include a plurality of torsion springs to secure the connector conduit relative to the organ. For example, as is shown in FIG. 32C, torsion spring 984 is positioned within the organ, and torsion spring 984A is positioned outside the wall of the organ, where the suture ring has been located for the other embodiments described above. When two torsion springs are used as expansion elements in this configuration, the torsion springs effectively compress the wall of the organ, thereby securing the connector conduit to the organ. It should be noted that is preferred that a flange element still be used in this configuration to prevent over-insertion of the connector conduit into the organ. In this arrangement, the flange element can be positioned on the pusher assembly of the applicator instead of on the connector conduit. As such, the second torsion spring 984A could be positioned immediately adjacent to the flange element. In addition, while FIG. 32C only shows the use of two torsion springs, three or more torsion springs may also be used. For example, depending on the thickness of the wall of the organ, torsion springs may reside inside the organ, within the wall of the organ, and/or outside of the organ, resulting in a ribbed effect that facilitates engagement of the connector conduit to the organ. In addition, torsion springs may be used in combination with balloons.

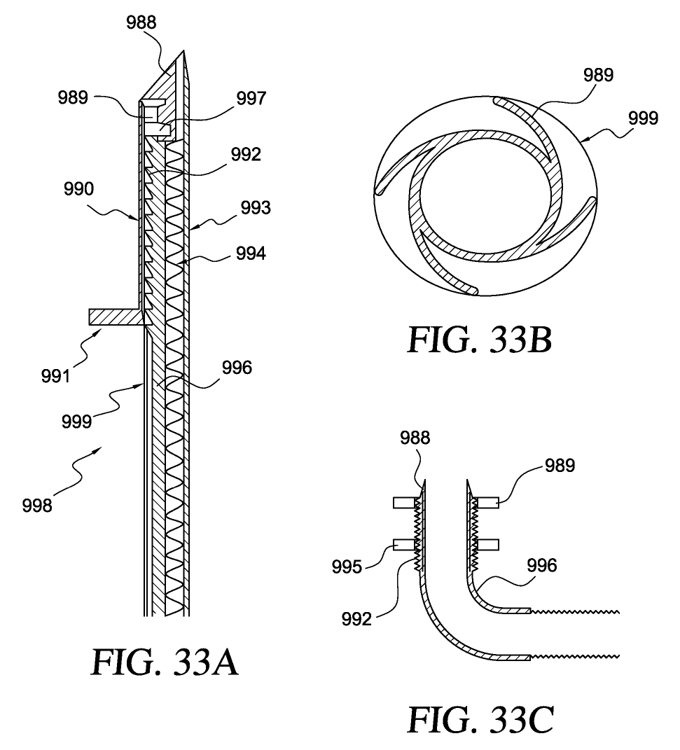

FIGS. 33A-33C illustrate an embodiment of the invention wherein a spiral spring is used as the retention means to retain the connector conduit securely within the organ. As is shown in the figures, a stent 988 is attached to connector conduit 998 for insertion through the wall of the organ. A spiral spring 989 is positioned, in a compressed state, in a circumferential groove 997 around stent 988, and is covered by an outer fabric sleeve 999, which is adapted to expand as the spiral spring expands from its compressed state to its expanded state. Spiral spring 989 is maintained in a compressed state by a smooth frame cover 990, which also includes a insertion stop 991. Spiral spring 989 should be formed of a strong material, such as a metal or plastic, such as PEEK.

During operation, the connector conduit is inserted through the wall of the organ, using cutter 993, until insertion stop 991 contacts the outer surface of the wall of the organ, as is described above with reference to the flange element. The spiral spring, which is initially in the compressed state, is inserted through the hole in the wall of the organ as the connector conduit is inserted through the hole in the wall of the organ. After the connector conduit has been inserted through the hole in the wall of the organ, with spiral spring 989 residing inside the organ, smooth frame cover 990 is withdrawn through the wall of the organ and spiral spring 989 is allowed to expand from the compressed state to an expanded state, thereby preventing the pressure in the organ from pushing the connector conduit back out the hole and preventing movement of the connector conduit relative to the wall of the organ. FIG. 33B shows an exemplary shape of spiral spring 989 when in an expanded state, and clearly shows the positioning of outer fabric sleeve 999 relative to spiral spring 989. After spiral spring 989 is expanded, a compression ring 995, which may be positioned circumferentially around the connector conduit on the outside of the organ, may be moved longitudinally along the surface of the connector conduit until being compressed down onto the external surface of the wall of the organ via a plurality of ratchet steps 992, which allows for a tight, compressed seal on the wall of the organ to be achieved between compression ring 995 and spiral spring 989. The engagement of the expanded spiral spring with the wall of the organ axially squeezes the wall of the organ between the expanded spiral spring and the sewing ring, thereby preventing any leakage of blood or fluids from within the organ around the engagement of the wall of the organ and the connector conduit.

Spiral spring 989 may also be used as a direct replacement for torsion spring 984 shown in FIG. 32A-32B. In this case, with reference to FIGS. 32A-32B, spiral spring 989 is positioned, in a compressed state, in a circumferential groove 987 around stent 981. An outer fabric sleeve 983 covers stent 981 and spiral spring 989. Spiral spring 989 is retained in a compressed state by a sheath 985 which is positioned over spiral spring 989 and outer fabric sleeve 983. During operation, the connector conduit is inserted through the wall of the organ until flange element 982 contacts the outer surface of wall 905, as is described above. After being properly positioned, with spiral spring 989 residing inside the organ, sheath 985 is withdrawn through the wall of the organ and spiral spring 989 is allowed to expand, thereby preventing the pressure in the organ from pushing the connector conduit back out the hole. As spiral spring 989 expands, the shape of outer fabric sleeve 983 conforms with the expanding shape of spiral spring 989. Thus, it is preferred that outer fabric sleeve 983 be formed into a shape that allows for the expansion of spiral spring 989 without any significant deformation or stretching. To facilitate this, outer fabric sleeve 983 may have folds or the like prior to expansion of spiral spring 989. FIG. 32B shows an exemplary shape of outer fabric 983 when spiral spring 989 is in an expanded state.

Although the present invention has been described in relation to particular embodiments thereof, many other variations and modifications and other uses will become apparent to those skilled in the art. It is preferred therefore, that the present invention be limited not by the specific disclosure herein, but only by the appended claims.

What is claimed is:

1. An apparatus for securing a connector conduit to a hollow organ and preventing blood loss from the hollow organ, the apparatus comprising:
a connector conduit operable to be inserted through a hole in a wall of the organ;
a flange element positioned on the connector conduit adapted to prevent over-insertion of the connector conduit; and
a retention means positioned on the connector conduit, the retention means being adapted to be engaged with the wall of the organ to prevent movement of the connector conduit relative to the wall of the organ after the connector conduit is inserted through the hole in the wall of the organ,
wherein the connector conduit is inserted through the hole in the wall of the organ until the flange element comes into contact with the wall of the organ, and
wherein the retention means is engaged with the wall of the organ after the connector conduit is inserted through the hole in the wall of the organ.

2. The apparatus for securing a connector conduit to a hollow organ and preventing blood loss from the hollow organ of claim 1, wherein the hole in the wall of the organ is formed by a hole forming element having a cutting element on a distal end thereof and being adapted for coupling with the connector conduit.

3. The apparatus for securing a connector conduit to a hollow organ and preventing blood loss from the hollow organ of claim 1, wherein the organ is a heart.

4. The apparatus for securing a connector conduit to a hollow organ and preventing blood loss from the hollow organ of claim 1, wherein the flange element is integrally formed on the connector conduit.

5. The apparatus for securing a connector conduit to a hollow organ and preventing blood loss from the hollow organ of claim 1, wherein the retention means comprises a plurality of retaining pins positioned circumferentially around the connector conduit, such that the retaining pins are inserted into the hole in the wall of the organ when the connector is inserted through the hole in the wall of the organ.

6. The apparatus for securing a connector conduit to a hollow organ and preventing blood loss from the hollow organ of claim 5, further comprising a means for causing the retaining pins to engage the wall of the organ to prevent movement of the connector conduit relative to the wall of the organ.

7. The apparatus for securing a connector conduit to a hollow organ and preventing blood loss from the hollow organ of claim 6, wherein the means for causing the retaining pins to engage the wall of the organ to prevent movement of the connector conduit relative to the wall of the organ comprises a plurality of skid elements and pull wires.

8. The apparatus for securing a connector conduit to a hollow organ and preventing blood loss from the hollow organ of claim 6, wherein the retaining pins are maintained in a passive state adjacent to an outer surface of the connector conduit until entering into engagement with the wall of the organ.

9. The apparatus for securing a connector conduit to a hollow organ and preventing blood loss from the hollow organ of claim 1, wherein the retention means comprises a plurality of prongs positioned circumferentially around the connector conduit such that the prongs, when in an initial passive state, are positioned outside of the organ after the connector conduit has been inserted through the hole in the wall of the organ.

10. The apparatus for securing a connector conduit to a hollow organ and preventing blood loss from the hollow organ of claim 9, wherein, after the connector conduit has been inserted through the hole in the wall of the organ, the prongs are adapted to be inserted through a plurality of holes in the flange element into the wall of the organ, thereby entering into engagement with the wall of the organ.

11. The apparatus for securing a connector conduit to a hollow organ and preventing blood loss from the hollow organ of claim 10, further comprising a prong installation element adapted to insert the prongs through the holes in the flange element into the wall of the organ, thereby causing the prongs to enter into engagement with the wall of the organ.

12. The apparatus for securing a connector conduit to a hollow organ and preventing blood loss from the hollow organ of claim 10, wherein the prongs have a curved shape that causes engagement of the prongs with the wall of the organ by the insertion of the prongs into the wall of the organ.

13. The apparatus for securing a connector conduit to a hollow organ and preventing blood loss from the hollow organ of claim 1, wherein the retention means comprises a balloon positioned on the connector conduit, such that the balloon is inserted through the hole in the wall of the organ as the connector conduit is inserted through the hole in the wall of the organ.

14. The apparatus for securing a connector conduit to a hollow organ and preventing blood loss from the hollow organ of claim 13, wherein the balloon is maintained in an initial deflated state until after the balloon and the connector conduit are inserted through the hole in the wall of the organ.

15. The apparatus for securing a connector conduit to a hollow organ and preventing blood loss from the hollow organ of claim 14, wherein, after the connector conduit has been inserted through the hole in the wall of the organ, the balloon is inflated from the initial deflated state to an expanded state, thereby entering into engagement with the wall of the organ and preventing movement of the connector conduit relative to the hole in the wall of the organ.

16. The apparatus for securing a connector conduit to a hollow organ and preventing blood loss from the hollow organ of claim 13, wherein the flange element is replaced with a second balloon positioned on the connector conduit such that, after insertion of the connector conduit through the hole in the wall of the organ, the two balloons are inflated, and the wall of the organ is compressed between the two balloons, thereby preventing movement of the connector conduit relative to the wall of the organ.

17. The apparatus for securing a connector conduit to a hollow organ and preventing blood loss from the hollow organ of claim 13, wherein the flange element is replaced with a torsion spring positioned on the connector conduit, such that, after insertion of the connector conduit through the hole in the wall of the organ, the balloon is inflated, and the wall of the organ is compressed between the torsion spring and the balloon, thereby preventing movement of the connector conduit relative to the wall of the organ.

18. The apparatus for securing a connector conduit to a hollow organ and preventing blood loss from the hollow organ of claim 1, wherein the retention means comprises a torsion spring positioned on the connector conduit, such that the torsion spring, when in an initial compressed state, is inserted through the hole in the wall of the organ as the connector conduit is inserted through the hole in the wall of the organ.

19. The apparatus for securing a connector conduit to a hollow organ and preventing blood loss from the hollow organ of claim 18, further comprising a sheath adapted to retain the torsion spring in a compressed state.

20. The apparatus for securing a connector conduit to a hollow organ and preventing blood loss from the hollow organ of claim 19, wherein, after the connector conduit has been inserted through the hole in the wall of the organ, the sheath is withdrawn from the hole in the wall of the organ, thereby allowing the torsion spring to expand from the initial compressed state to an expanded state, thereby entering into engagement with the wall of the organ and preventing movement of the connector conduit relative to the wall of the organ.

21. The apparatus for securing a connector conduit to a hollow organ and preventing blood loss from the hollow organ of claim 19, wherein the flange element is replaced with a second torsion spring positioned on the connector conduit such that, after insertion of the connector conduit through the hole in the wall of the organ, and withdrawal of the sheath from the wall of the organ, the two torsion springs are in their respective expanded states, and the wall of the organ is compressed between the two torsion springs, thereby preventing movement of the connector conduit relative to the wall of the organ.

22. The apparatus for securing a connector conduit to a hollow organ and preventing blood loss from the hollow organ of claim 19, wherein the flange element is replaced by a plurality of torsion springs positioned on the connector conduit such that, after insertion of the connector conduit through the hole in the wall of the organ, and withdrawal of the sheath from the wall of the organ, at least one torsion spring resides inside the organ, at least one torsion spring resides within the wall of the organ, and at least one torsion spring resides outside of the organ, thereby compressing the wall of the organ between the two torsion springs and preventing movement of the connector conduit relative to the wall of the organ.

23. The apparatus for securing a connector conduit to a hollow organ and preventing blood loss from the hollow organ of claim 19, wherein the flange element is replaced with a balloon positioned on the connector conduit such that, after insertion of the connector conduit through the hole in the wall of the organ, withdrawal of the sheath from the wall of the organ, and inflation of the balloon, the torsion spring is in its expanded state, the balloon is in its inflated state, and the wall of the organ is compressed between the torsion spring and the balloon, thereby preventing movement of the connector conduit relative to the wall of the organ.

24. The apparatus for securing a connector conduit to a hollow organ and preventing blood loss from the hollow organ of claim 1, wherein the retention means comprises a spiral spring positioned on the connector conduit, such that the spiral spring, when in an initial compressed state, is inserted through the hole in the wall of the organ as the connector conduit is inserted through the hole in the wall of the organ.

25. The apparatus for securing a connector conduit to a hollow organ and preventing blood loss from the hollow organ of claim 24, further comprising a smooth frame cover adapted to retain the spiral spring in a compressed state.

26. The apparatus for securing a connector conduit to a hollow organ and preventing blood loss from the hollow organ of claim 25, wherein, after the connector conduit has been inserted through the hole in the wall of the organ, the smooth frame cover is withdrawn from the hole in the wall of the organ, thereby allowing the spiral spring to expand from the compressed state to an expanded state, thereby entering into engagement with the wall of the organ and preventing movement of the connector conduit relative to the wall of the organ.

27. The apparatus for securing a connector conduit to a hollow organ and preventing blood loss from the hollow organ of claim 24, wherein the flange element is replaced by a compression ring, which is positioned circumferentially around the connector conduit on the outside of the organ, such that, after the connector conduit is inserted through the hole in the wall of the organ, the spiral spring expands from the compressed state to an expanded state, and the compression ring is moved longitudinally along the surface of the connector conduit along one or more ratchet steps formed on the surface of the connector conduit towards the wall of the organ, thereby compressing the wall of the organ between the spiral spring and the compression ring, and preventing movement of the connector conduit relative to the wall of the organ.

28. A method for securing a connector conduit to a hollow organ and preventing blood loss from the hollow organ, the method comprising:
forming a hole in a wall of the organ;
inserting a connector conduit through the hole in the wall of the organ until a flange element comes into contact with the wall of the organ, the flange element being positioned on the connector conduit; and
engaging a retention means with the wall of the organ to prevent movement of the connector conduit relative to the wall of the organ, the retention means being positioned on the connector conduit.

29. The method for securing a connector conduit to a hollow organ and preventing blood loss from the hollow organ of claim 28, wherein the hole in the wall of the organ is formed by a hole forming element having a cutting element on a distal end thereof and being adapted for coupling with the connector conduit.

30. The method for securing a connector conduit to a hollow organ and preventing blood loss from the hollow organ of claim 28, wherein the organ is a heart.

31. The method for securing a connector conduit to a hollow organ and preventing blood loss from the hollow organ of claim 28, wherein the flange element is integrally formed on the connector conduit.

32. The method for securing a connector conduit to a hollow organ and preventing blood loss from the hollow organ of claim 28, wherein the retention means comprises a plurality of retaining pins positioned circumferentially around the connector conduit, such that the retaining pins are inserted into the hole in the wall of the organ when the connector is inserted through the hole in the wall of the organ.

33. The method for securing a connector conduit to a hollow organ and preventing blood loss from the hollow organ of claim 32, wherein the step of engaging comprises causing the retaining pins to penetrate the wall of the organ to prevent movement of the connector conduit relative to the wall of the organ.

34. The method for securing a connector conduit to a hollow organ and preventing blood loss from the hollow organ of claim 33, wherein the retaining pins are maintained in a passive state adjacent to an outer surface of the connector conduit until entering into engagement with the wall of the organ.

35. The method for securing a connector conduit to a hollow organ and preventing blood loss from the hollow organ of claim 28, wherein the retention means comprises a plurality of prongs positioned circumferentially around the connector conduit such that the prongs, when in an initial passive state, are positioned outside of the organ after the connector conduit has been inserted through the hole in the wall of the organ.

36. The method for securing a connector conduit to a hollow organ and preventing blood loss from the hollow organ of claim 35, wherein the step of engaging comprises inserting the prongs through a plurality of holes in the flange element into the wall of the organ, thereby engaging the wall of the organ and preventing movement of the connector conduit relative to the wall of the organ.

37. The apparatus for securing a connector conduit to a hollow organ and preventing blood loss from the hollow organ of claim 28, wherein the retention means comprises a balloon positioned on the connector conduit, such that the balloon is inserted through the hole in the wall of the organ as the connector conduit is inserted through the hole in the wall of the organ.

38. The method for securing a connector conduit to a hollow organ and preventing blood loss from the hollow organ of claim 37, wherein the balloon is maintained in an initial deflated state until after the balloon and the connector conduit are inserted through the hole in the wall of the organ.

39. The method for securing a connector conduit to a hollow organ and preventing blood loss from the hollow organ of claim 38, wherein step of engaging comprises inflating the balloon from the initial deflated state to an expanded state, thereby engaging the wall of the organ and preventing movement of the connector conduit relative to the wall of the organ.

40. The method for securing a connector conduit to a hollow organ and preventing blood loss from the hollow organ of claim 37, wherein the flange element is replaced with a second balloon positioned on the connector conduit such that the step of engaging comprises inflating both of the balloons to compress the wall of the organ between the two balloons, thereby preventing movement of the connector conduit relative to the wall of the organ.

41. The apparatus for securing a connector conduit to a hollow organ and preventing blood loss from the hollow organ of claim 37, wherein the flange element is replaced with a torsion spring positioned on the connector conduit, such that the step of engaging comprises inflating the balloon and expanding the torsion spring from a compressed state to an expanded state to compress the wall of the organ between the torsion spring and the balloon, thereby preventing movement of the connector conduit relative to the wall of the organ.

42. The method for securing a connector conduit to a hollow organ and preventing blood loss from the hollow organ of claim 28, wherein the retention means comprises a torsion spring positioned on the connector conduit, such that the torsion spring, when in an initial compressed state, is inserted through the hole in the wall of the organ as the connector conduit is inserted through the hole in the wall of the organ.

43. The method for securing a connector conduit to a hollow organ and preventing blood loss from the hollow organ of claim 42, wherein the torsion spring is retained in the initial compressed state by a sheath.

44. The method for securing a connector conduit to a hollow organ and preventing blood loss from the hollow organ of claim 43, wherein the step of engaging comprises withdrawing the sheath from the hole in the wall of the organ, thereby allowing the torsion spring to expand from the initial compressed state to an expanded state and enter into engagement with the wall of the organ, thereby preventing movement of the connector conduit relative to the wall of the organ.

45. The method for securing a connector conduit to a hollow organ and preventing blood loss from the hollow organ of claim 43, wherein the flange element is replaced with a second torsion spring positioned on the connector conduit such that the step of engaging comprises withdrawing the sheath from the wall of the organ, thereby allowing the torsion springs to expand from the compressed state to the expanded states to compress the wall of the organ between the two torsion springs, thereby preventing movement of the connector conduit relative to the wall of the organ.

46. The method for securing a connector conduit to a hollow organ and preventing blood loss from the hollow organ of claim 43, wherein the flange element is replaced by a plurality of torsion springs positioned on the connector conduit such that the step of engaging comprises withdrawing the sheath from the wall of the organ, wherein at least one torsion spring resides inside the organ, at least one torsion spring resides within the wall of the organ, and at least one torsion spring resides outside of the organ, thereby compressing the wall of the organ between the two torsion springs and preventing movement of the connector conduit relative to the wall of the organ.

47. The method for securing a connector conduit to a hollow organ and preventing blood loss from the hollow organ of claim 43, wherein the flange element is replaced with a balloon positioned on the connector conduit such that the step of engaging comprises withdrawing the sheath from the wall of the organ, thereby allowing the torsion spring to expand from the compressed state to the expanded state, and inflating the balloon, to compress the wall of the organ between the torsion spring and the balloon, thereby preventing movement of the connector conduit relative to the wall of the organ.

48. The apparatus for securing a connector conduit to a hollow organ and preventing blood loss from the hollow organ of claim 28, wherein a spiral spring is positioned on the connector conduit, such that the spiral spring, when in an initial compressed state, is inserted through the hole in the wall of the organ as the connector conduit is inserted through the hole in the wall of the organ.

49. The method for securing a connector conduit to a hollow organ and preventing blood loss from the hollow organ of claim 48, further comprising a smooth frame cover adapted to retain the spiral spring in a compressed state.

50. The method for securing a connector conduit to a hollow organ and preventing blood loss from the hollow organ of claim 49, wherein the step of engaging comprises withdrawing the smooth frame cover from the hole in the wall of the organ, thereby allowing the spiral spring to expand from the compressed state to an expanded state, thereby entering into engagement with the wall of the organ and preventing movement of the connector conduit relative to the wall of the organ.

51. The apparatus for securing a connector conduit to a hollow organ and preventing blood loss from the hollow organ of claim 49, wherein the flange element is replaced by a compression ring, which is positioned circumferentially around the connector conduit on the outside of the organ, such that the step of engaging comprises withdrawing the smooth frame cover to allow the spiral spring to expand from the compressed state to an expanded state, and moving the compression ring longitudinally along the surface of the connector conduit along one or more ratchet steps formed on the surface of the connector conduit towards the wall of the organ, thereby compressing the wall of the organ between the spiral spring and the compression ring, and preventing movement of the connector conduit relative to the wall of the organ.
